(19) United States
(12) Patent Application Publication
(10) Pub. No.: US 2024/0124617 A1
(43) Pub. Date: Apr. 18, 2024

(54) COMPOSITIONS COMPRISING EXOPOLYSACCHARIDES AND USES THEREOF

(71) Applicant: DANSTAR FERMENT AG, Zug (CH)

(72) Inventor: Frédéric DURMONT, Saint Moritz (CH)

(21) Appl. No.: 18/546,073

(22) PCT Filed: Feb. 10, 2022
(86) PCT No.: PCT/EP2022/053270

§ 371 Date: Aug. 10, 2023

(30) Foreign Application Priority Data
Feb. 11, 2021 (EP) 21305183.2

(51) Int. Cl. C08B 37/00; A01N 43/16; A61K 31/715; A61P 31/16; A61P 31/20; C12P 19/04

(52) U.S. Cl. CPC C08B 37/006; A01N 43/16; A61K 31/715; A61P 31/16; A61P 31/20; C12P 19/04

(57) ABSTRACT

The present disclosure relates to exopolysaccharides produced by marine bacteria, compositions thereof, the use of aid compositions, and a method for attenuating the virulence of a microbial pathogen infection, either bacterial or viral infections, by inhibiting or reducing colonization of said microbial pathogens onto biological and/or non-biological surfaces.

### TECHNOLOGICAL FIELD

The present invention relates to exopolysaccharides (EPS) originating from a marine bacteria, compositions thereof, non-therapeutic uses of said EPS and compositions, and therapeutic uses of said EPS and compositions. The present invention also relates to a process for obtaining said EPS. The present invention further relates to a nasal delivery system comprising at least one isolated exopolysaccharide and therapeutic uses of said nasal delivery system. The present invention also concerns a method of formulating a nasal composition.

### BACKGROUND

Infectious diseases are usually caused by microorganisms that invade the body and spread. There are many types of infectious organisms, also called pathogenic microorganisms. Here are some examples of how these microorganisms can invade the body: through the mouth, eyes or nose, through sexual contact, through wounds or bites, through contaminated medical devices.

People are mainly infected by such microorganisms by ingesting contaminated water or eating contaminated food. They may inhale spores or dust, or inhale contaminated droplets from someone else's cough or sneeze. People may also handle contaminated objects (such as a door handle) or come into direct contact with a contaminated person and then touch their eyes, nose or mouth.

Some microorganisms also spread into body fluids, such as blood, semen and stool. For example, they can invade the body through sexual contact with an infected partner. Human and animal bites and other wounds that pierce the skin can allow pathogenic microorganisms to invade the body.

Pathogenic microorganisms can also adhere to medical devices implanted in the body (such as catheters, artificial joints or artificial heart valves). They may be present on the device when implanted, if the device has been accidentally contaminated. Infectious agents from other locations can also spread through the bloodstream and attach to a device that has already been implanted. Because the implanted device lacks natural defenses, such microorganisms can easily grow and spread, causing disease.

After invading the human body, pathogenic microorganisms must multiply or spread to cause an infection. After multiplication or spreading begins, three scenarios can occur: 1) The microorganisms continue to multiply and overwhelm the host organism's defenses. 2)

A state of equilibrium is reached, causing a chronic infection. 3) The host organism, with or without medical treatment, destroys and eliminates the invasive microorganisms.

The effectiveness of existing solutions (i.e., antibiotics, antivirals) to get rid of pathogenic infections depends on the fast recognition of the pathogen followed by the application of a pathogen-suppressing composition prior to microorganism irreversible adhesion to a surface. It would be highly desirable to be provided with a different approach limiting microbial pathogen irreversible adhesion onto surface, allowing a reduction of treatment resistance phenomena and therefore their impact on associated healthcare costs.

### SUMMARY OF THE INVENTION

According to a first aspect, the present disclosure provides an isolated exopolysaccharide (EPS) having a weight average molecular weight ranging from 40 to 4000 kDa, wherein the EPS is obtained or obtainable by fermentation of a marine bacteria. In an embodiment, the isolated EPS has a weight average molecular weight (Mw) ranging from 40 to 2000 kDa, 40 to 1400 kDa, 40 to 1000 kDa, 40 to 500 kDa, 40 to 400 kDa, 40 to 300 kDa, 40 to 200 kDa, 40 to 150 kDa, or 40 to 100 kDa. In a preferred embodiment, the isolated EPS has a Mw ranging from 40 to 150 kDa. In an embodiment, the isolated EPS has a Mw ranging from 40 to 150 kDa and optionally a number average molecular weight (Mn) ranging from 26 to 100 kDa and/or a polydispersity index (Mw/Mn) ranging from 1.2 to 1.8. In an embodiment, an isolated EPS as described herein may comprise 30-90% of neutral glycosyl unit, 10-70% of amino glycosyl units, and 0-15% of acidic glycosyl unit with respect to the total number of glycosyl units of said EPS. In another embodiment, an isolated EPS as described herein may comprise mannose, galactose, glucose, N-acetyl galactosamine and N-acetyl glucosamine. In some further embodiment, an isolated EPS as described herein may be substantially free of ribose, arabinose, rhamnose, fructose and/or fucose. In another embodiment, the marine bacteria may be a gram-positive thermophilic bacteria, preferably Bacillus licheniformis, more preferably Bacillus licheniformis LP-T14 which was deposited with The National Collection of Industrial, Food and Marine Bacteria (NCIMB) on 27 Jan. 2020 under deposit number NCIMB 43557. In another embodiment, an isolated EPS as described herein may substantially lack the ability to induce an immune response.

According to a second aspect, the present disclosure provides a process for obtaining an isolated exopolysaccharide (EPS) having a weight average molecular weight (Mw) ranging from 40 to 4000 kDa comprising the steps of: culturing a marine bacteria in a first culture medium to obtain a cultured marine bacteria; fermenting the cultured marine bacteria in a fermentation medium; heat treating the fermentation medium; centrifuging the fermentation medium to obtain a supernatant; and filtering the supernatant to obtain the isolated EPS. The isolated EPS may be an isolated EPS as described herein. In an embodiment, the marine bacteria may be a gram-positive thermophilic bacteria, preferably Bacillus licheniformis, more preferably Bacillus licheniformis LP-T14 which was deposited with The National Collection of Industrial, Food and Marine Bacteria (NCIMB) on 27 Jan. 2020 under deposit number NCIMB 43557. In an embodiment, the fermentation medium may comprise sea salts (40 g/L), tryptone (6 g/L), yeast extract (6 g/L), antifoam (0.33 mL/L), dextrose (12 g/L) and deionized H2O (qsp.). In an embodiment, the fermentation may be performed at pH 5-8 for a period of 20-40 h at 40° C., under 30-50% oxygenation. In an embodiment, the heat treatment of fermentation medium may be performed by heating the fermentation medium for 1 h at 85° C. In an embodiment, the centrifugation of the fermentation medium may be performed at 14000 g using a disk stack centrifuge with a flow rate of 200-800 L/h. In an embodiment, the filtration of supernatant comprises successive filtration using 1.60 to 0.22 μm filtration steps and/or ultrafiltration using 10 to 100 kDa cut-off filter cartridges. In an embodiment, the process of the present disclosure may further comprise free-drying the isolated EPS to obtain a free-dried EPS. In an embodiment, the free-drying is performed at −20° C. for at least 16 hours

According to a third aspect, the present disclosure provides a composition which comprises at least one isolated EPS as described herein and a suitable carrier. The composition of the present invention may be an oral composition, a nasal composition, a topical composition, a transdermal composition, an ophthalmic composition or a composition formulated for applying on a medical device.

According to a fourth aspect, the present disclosure provides a nasal delivery system which comprises an at least one isolated EPS as described herein or at least one isolated EPS obtained or obtainable by the process described herein. In an embodiment, the nasal delivery system may deliver the at least one isolated EPS as nose drops, a liquid spray, a dried spray, a gel or an ointment. In another embodiment, the at least one isolated EPS may be formulated in a composition that further comprises a saline solution. In another embodiment, a nasal delivery system as described herein may be for treating and/or preventing a microbial pathogen infection in a subject in need thereof. In another embodiment, a nasal delivery system as described herein may be for use in treating and/or preventing a microbial pathogen infection in a subject in need thereof. In some embodiment, a nasal delivery system as described herein may be for attenuating the virulence of a microbial pathogen by inhibiting or reducing colonization by the microbial pathogen of the nasal cavity of a subject in need thereof. In some embodiment, a nasal delivery system as described herein may be for use in attenuating the virulence of a microbial pathogen by inhibiting or reducing colonization by the microbial pathogen of the nasal cavity of a subject in need thereof.

According to a fifth aspect, the present disclosure provides a non-therapeutic use of an isolated EPS as described herein or an isolated EPS obtained or obtainable by the process described herein or a composition as described herein for treating a non-biological surface to prevent or reduce the colonization of the surface by a microbial pathogen. In an embodiment, the surface may be a surface of a medical device.

According to a sixth aspect, the present disclosure provides an isolated EPS as described herein or an isolated EPS obtained or obtainable by the process described herein, a composition as described herein or a nasal delivery system as described herein for use in a method for treating and/or preventing a microbial pathogen infection in a subject in need thereof.

According to a seventh aspect, the present disclosure provides the use of an isolated EPS as described herein or an isolated EPS obtained or obtainable by the process described herein, a composition as described herein or a nasal delivery system as described herein for the manufacture of a medicament for the treatment and/or prevention of a microbial pathogen infection.

According to an eighth aspect, the present disclosure provides an isolated EPS as described herein or an isolated EPS obtained or obtainable by the process described herein, a composition as described herein, or a nasal delivery system for use in a method for attenuating the virulence of a microbial pathogen.

According to a ninth aspect, the present disclosure provides the use of an isolated EPS as described herein or an isolated EPS obtained or obtainable by the process described herein, a composition as described herein or a nasal delivery system as described herein for the manufacture of a medicament for attenuating the virulence of a microbial pathogen.

According to a tenth aspect, the present disclosure provides a method of treating and/or preventing a microbial pathogen infection in a subject in need thereof, the method comprising administering to the subject an effective amount of an isolated EPS as described herein or an isolated EPS obtained or obtainable by the process described herein or a composition as described herein.

According to a eleventh aspect, the present disclosure provides a method for attenuating the virulence of a microbial pathogen infection in a subject in need thereof, the method comprising the administration of an effective amount of an isolated EPS as described herein or an isolated EPS obtained or obtainable by the process described herein or a composition as described herein to the subject.

According to a twelfth aspect, the present disclosure provides a method of formulating a nasal composition for attenuating the virulence of a microbial pathogen infection by inhibiting or reducing colonization by a microbial pathogen within the nasal cavity, wherein the method comprises the step of mixing at least one isolated exopolysaccharide (EPS) originating from a marine bacteria with a saline solution to obtain a nasal composition comprising salt at a concentration of 0.1% to 10% w/w. In an embodiment, the at least one isolated EPS may be an isolated EPS according to the present disclosure. In a preferred embodiment, the obtained nasal composition comprises salt at a concentration of 0.5% to 5% w/w. More preferably, the obtained nasal composition comprises salt at a concentration of 0.7% to 3% w/w. In an embodiment, the obtained composition comprises a salt concentration of about 0.9% w/w. In an embodiment, the obtained composition comprises a salt concentration of about 2.2% w/w. In an embodiment, the obtained composition comprises a salt concentration of about 2.7% w/w.

According to any of the isolated EPS for use according to the present disclosure, the composition for use according to the present disclosure or the methods of the present disclosure, at least one isolated EPS or composition as described herein may be administered to the subject prior, during, and/or after the microbial pathogen infection, thereby preventing, inhibiting, or reducing colonization of the microbial pathogen at least one biological tissue of the subject and/or the internalization of the microbial pathogen. In an embodiment, the at least one biological tissue may be selected from the oral cavity, the nasal cavity, the respiratory tract, the throat, the ears, the ophthalmic region, the urogenital tract, the skin, the scalp, the hairs, the nails, and combinations thereof. In another embodiment, the microbial pathogen may be a bacterial pathogen and administration of the isolated EPS or the composition to the subject attenuates the virulence of the bacterial pathogen infection by reducing or inhibiting early bacterial biofilm formation and/or disrupting early bacterial biofilm. In another embodiment, the microbial pathogen may be a viral pathogen and administration of the isolated EPS or the composition to the subject attenuates the virulence of a viral pathogen infection by reducing the mortality of subject cells infected with the viral pathogen as compared to equivalent untreated cells, by preventing or reducing the release of virions from inoculated subject cells and/or by preventing or reducing the infection of uninfected adjacent subject cells.

According to any of the nasal delivery systems of the present disclosure, the non-therapeutic uses of the present disclosure, the isolated EPS for use according to the present disclosure, compositions for use according to the present disclosure, or the methods of the present disclosure, the microbial pathogen may be at least one of a bacterial pathogen, a viral pathogen, or a fungal pathogen. In an embodiment, said at least one bacterial pathogen may be selected from the Cutibacterium, Haemophilus, Klebsiella, Moraxella, Pseudomonas, Staphylococcus and Streptococcus genera. In some embodiment, the at least one of bacterial pathogen may be a Cutibacterium acnes, Haemophilus influenzae, Klebsiella pneumoniae, Moraxella catharalis, Pseudomonas aeruginosa, Staphylococcus aureus, Streptococcus pneumoniae or Streptococcus mutans species. In an embodiment, the at least one bacterial pathogen may be selected from Haemophilus influenzae, Klebsiella pneumoniae, Pseudomonas aeruginosa, Staphylococcus aureus, Streptococcus pneumoniae, and/or Streptococcus mutans species. In another embodiment, said at least one viral pathogen may be selected from Influenza A, Influenza A subtype H1N1/2009/pdm09, Influenza A subtype H1, Influenza A subtype H3, Influenza B (Orthomyxovirus), Coronavirus 229E, Coronavirus HKU1, Coronavirus NL63, Coronavirus 0C43, SARS-CoV-2 (Coronavirus), Parainfluenza Virus 1, Parainfluenza Virus 2, Parainfluenza Virus 3, Parainfluenza Virus 4, Respiratory Syncytial Virus A/B, Human Metapneumovirus NB (Paramyxovirus), Adenovirus or Rhinovirus/Enterovirus (Picornavirus). In a further embodiment, the at least on viral pathogen may be selected from Coronavirus OC43, adenovirus, and/or Rhinovirus/Enterovirus (Picornavirus).

The amount of the at least one EPS in the composition of the present disclosure or the composition used according to the methods and uses of the present disclosure, based on the total weight of the composition, may be in the range of 0.00005-0.5% w/w, preferably 0.0001-0.1% w/w, more preferably 0.0005-0.05% w/w.

### FIGURES

FIG. 1 represents the step-by-step process used for the production of the EPS of the present disclosure.

FIG. 2 shows the biofilm formation (%) of various bacterial pathogens at different EPS concentrations. Biofilm formation was normalized to untreated bacterial cells Control). Errors bars represent the standard deviation of two independent experiments (N =2) carried out in triplicate (n=3). *p<0.05 vs. Control; **p<0.01 vs. Control;

FIG. 3 shows the antibacterial effect evaluation of increasing EPS concentration on various bacterial pathogens culture growth in two independent experiments (N=2) carried out in triplicate (n=3), comparing OD600 values, mean±SD. The growth culture in absence of EPS was used as control;

FIG. 4 shows the viability of HNEpC cultured with increasing concentrations of EPS during 2 and 24 h. Viability of TO-PRO3 negative cells after 2 and 24 h culture in complete medium was used as control (C). Errors bars represent the standard deviation of one experiment (N =1) carried out in triplicate (n=3);

FIG. 5 shows the reduction of adhesion on HNEpC of P. aeruginosa and S. aureus of in the presence of increasing concentration of EPS. Biofilm formation (Cell adhesion) was normalized to untreated cells (Control). Errors bars represent the standard error of the mean of 2 to 4 independent experiments (N=2-4) carried out in triplicate (n =3). **p<0.01 vs. Control;

FIG. 6 shows the emulsifying Activity Index after mixing various concentrations of EPS solution with vegetable oil followed by a 24 hours-rest period. Error bars represents the standard deviation of three independent experiments (N=3);

FIG. 7 shows the viability of HNEpC infected with Adenovirus or Rhinovirus, or treated with EPS (400 μg/mL) for 2 h before or after high dosage virus infection. Cells cultured for 48 h culture in BEBM complete medium were used as positive control, whereas virus inoculated cells without EPS treatment were used as negative control. Values represent mean of TO-PRO-3 negative (viable cells)±SD of two independent experiments (N=2) performed in triplicates (n=3);

FIG. 8A and FIG. 8B show the viability of HNEpC infected with Adenovirus or Rhinovirus, respectively, or treated with EPS (200 μg/mL) for 2 h before or after high dosage virus infection. Cells cultured for 48 h culture in BEBM complete medium were used as positive control, whereas virus inoculated cells without EPS treatment were used as negative control. Values represent mean of TO-PRO- 3 negative (viable cells)±SD of two independent experiments (N=2) performed in triplicates (n=3);

FIG. 9 shows the viability of HNEpC infected with Adenovirus or Rhinovirus, or pre-treated with EPS (400 μg/mL) for 2h before infection, or post-treated by adding EPS for 5 days or post-treated by replacing EPS every day for 5 days. Viability of TO-PRO-3 negative cells after 5 days of culture in BEBM complete medium was used as control. Errors bars represent the standard deviation of two independent experiments (N=2) carried out in duplicates (n=2);

FIG. 10A and FIG. 10B show the viability of HNEpC infected with Adenovirus or Rhinovirus, respectively, or pre- treated with EPS (200 μg/mL) for 2 h before infection, or post-treated by adding EPS for 5 days or post- treated by replacing EPS every day for 5 days. Viability of TO-PRO-3 negative cells after 5 days of culture in BEBM complete medium was used as control. Errors bars represent the standard deviation of two independent experiments (N=2) carried out in triplicates (n=3);

FIG. 11 shows the viability of HNEpC infected with Coronavirus (1 TCID50 or 0.001 TCID50), or pre- treated with EPS (200-400 μg/mL) for 2h before infection, or post-treated by adding 1 EPS dose for 5 days. Viability of TO-PRO-3 negative cells after 5 days of culture in BEBM complete medium was used as control. Errors bars represent the standard deviation of two independent experiments (N=2) carried out in triplicates (n=3);

FIG. 12A and FIG. 128 show the proportion of IL-8-producing HNEpC and IL-6-producing HNEpC, respectively, upon 24 h of stimulation with the EPS sample (400 μg/mL). Cells left unstimulated or stimulated with IL-1β were employed as negative and positive controls, respectively. Data represent results from three independent experiments (N=3), Error bars represent SD;

FIG. 13A and FIG. 13B show A) the expression of activation/maturation molecules (CD83, CD40 and CD25) on dendritic cells (DC) cultured with the EPS sample (400 μg/mL) for 24 h. Cells left unstimulated or stimulated with lipopolysaccharides (LPS, 1 μg/mL) were employed as negative and positive controls, respectively. Bars represent mean value±SEM of MFI of the indicated molecules. PBMC derived DC from two different donors were used for experiments in duplicate (N=2, n=2). Error bars represents SD; B) the proportion of IL-8-producing DC and IL-6-producing DC upon 24 h of stimulation with the EPS sample (400 μg/mL). Cells left unstimulated or stimulated with LPS were employed as negative and positive controls, respectively. Data represent results from three independent experiments (N=2, n=2), Error bars represent SD.

### DETAILED DESCRIPTION

A novel pathogenic infection management approach is proposed in the present disclosure, based on measures which consist in reducing or preventing pathogen adhesion onto surfaces. Thus, chemical components released by marine bacteria isolated from the shallow hydrothermal vents in the sea surrounding the Panarea island (Italy) were studied to investigate their ability to interfere, control and/or inhibit the reversible adhesion step of pathogenic microorganisms, such as bacteria, fungi and viruses onto biological and non-biological surfaces.

A bacteria from marine sources, namely Bacillus licheniformis LP-T14 which was deposited with the NCIMB (The National Collection of Industrial, Food and Marine Bacteria, NCIMB Ltd. Ferguson Building, Craibstone Estate, Bucksburn, Aberdeen, AB21 9YA, Scotland, UK) on 27 Jan. 2020 by Danstar Ferment A. G. (Poststrasse 30, Zug, 6300, Switzerland) under deposit number NCIMB 43557, has the uncommon ability to grow in extreme environmental conditions, such as high temperature (50° C.), salt water (conductivity of 42.90 mS·cm⁻¹), pH 5.4, high concentration of hydrogen sulfide and heavy metals, and the exopolysaccharides secreted by such marine strain were found to have unique physicochemical properties.

Thus, the disclosure herein demonstrates the ability of a new exopolysaccharide (EPS) released by the marine strain Bacillus licheniformis LP-T14 in attenuating the virulence of a pathogen infection by preventing and/or reducing pathogens adhesion on treated surfaces, when compared to untreated surface.

The present disclosure provides an EPS, a composition thereof, a use of the composition and a method for attenuating a virulence of a microbial pathogen infection. In the present disclosure, the virulence of a microbial pathogen infection is attenuated by inhibiting or reducing colonization of microbial pathogens responsible of the infection on biological and/or non-biological surfaces.

In the context of the present disclosure, the term “virulence” as used herein means the ability of a microbe (i.e., bacteria, virus, yeasts, fungi) to multiply and/or spread into a living organism, thereby overwhelming bodily defensive mechanisms. Therefore, when used as a prophylactic and/or therapeutic treatment, the composition of the present disclosure (indirectly) supports the host's immune system so that it can effectively defend itself against any type of pathogenic microbial infection.

Still in the context of the present disclosure, the expression “inhibiting or reducing colonization” refers to any action related to the composition disclosed herein having the effect of disturbing the installation of pathogenic bacteria on a surface and thus their multiplication, namely, without being limited to: inhibition or reduction of the formation of early biofilm, disruption or degradation (partial or total) of an early biofilm, disruption or degradation (partial or total) of an advanced biofilm.

In the context of the present disclosure, the expression “inhibiting or reducing colonization” refers to any action related to the composition disclosed herein having the effect of locally perturbing the establishment of viruses in a living organism. Thus, the composition has the effect of, without being limited to, partially or totally preventing the internalization of viruses within a living cell and/or limiting the spreading of virions from infected cells to adjacent living cells.

### Exopolysaccharides and Compositions Thereof

Bacteria from marine sources have the uncommon ability to grow in extreme environmental conditions, such as high temperature, salt water, high concentration of hydrogen sulfide and heavy metals, and the exopolysaccharides (EPS) produced by marine species were found to have unique physical properties and molecular structure. Marine bacterial EPS have therefore wide range of biotechnological, medical and pharmaceutical applications, thanks to their peculiar characteristics, such as water solubility, biodegradability, biocompatibility, bioadhesivity, heat resistance, swelling or gelling power.

EPS are polysaccharides which, after being produced by bacterial fermentation, are released into the culture medium. They are composed of monosaccharides that are linked to each other via glycosidic linkage. They may consist of one or more types of monosaccharides and may observe repeating units of monosaccharide blocks, ordered or randomly distributed. The EPS skeleton can be either linear or branched, in which case the branching arms can be of different lengths and include either the same or different monosaccharide units.

In one embodiment, the at least one isolated EPS is obtained or obtainable by fermentation of marine bacteria and an acceptable carrier thereof. In another embodiment, the composition of the present disclosure comprises at least one isolated EPS obtained or obtainable by fermentation of a gram-positive thermophilic bacteria and an acceptable carrier thereof. In another embodiment, the composition of the present disclosure comprises at least one isolated EPS obtained or obtainable by fermentation of a bacteria belonging to the Bacillus genus and an acceptable carrier thereof. In another embodiment, the composition of the present disclosure comprises at least one isolated EPS obtained or obtainable by fermentation of a bacteria belonging to the Bacillus licheniformis species and an acceptable carrier thereof. In yet another embodiment, the composition of the present disclosure comprises at least one isolated EPS obtained or obtainable by fermentation of a specific bacterial strain, the extremophile thermophilic marine Bacillus licheniformis LP-T14 deposited at the National Collection of Industrial, Food and Marine Bacteria (NCIMB) under NCIMB 43557 and an acceptable carrier thereof.

In an embodiment, the fermentation of marine bacteria is performed in a fermentation medium comprising sea salts (40 g/L), tryptone (6 g/L), yeast extract (6 g/L), antifoam (0.33 mL/L), dextrose (12 g/L) and deionized H₂O (qsp.) at pH 5-8, for a period of 20-40 h at 40° C., under 30-50% oxygenation. At the end of the fermentation, both marine bacteria and proteins from said marine bacteria can be inactivated by heating the fermentation medium for 1 h at 85° C., prior to EPS isolation.

The term “isolated” should be considered to mean materially removed from its original environment in which it is naturally produced, for example, in this instance a fermentation medium. The removed material is typically purified from the environment in which it was produced. In an embodiment, the EPS in an isolated form ideally does not contain any significant amounts of the bacterial strain, which can be in some embodiments partially or totally inactivated. In some another embodiment, the EPS in an isolated form contains negligible amounts of bacterial strains. In a further embodiment, the EPS in an isolated form contains at most 10¹ CFU/g of EPS, at most 10 2 CFU/g of EPS, at most 10³ CFU/g of EPS, at most 10⁴ CFU/g of EPS, or at most 10⁵ CFU/g. In an embodiment, the EPS in isolated form lacks a detectable amount of proteins from the bacterial strain used to produce it, which can be in some embodiments partially or totally inactivated, when analyzed by Lowry assay (Example 4). In some embodiment, the EPS in an isolated form contains negligible amount of proteins from the bacterial strain used to produce it, when analyzed by Lowry assay (Example 4). In some further embodiment, the EPS in an isolated form contains at most 0.1% w/w, at most 0.5% w/w, at most 1% w/w, at most 2% w/w, at most 3% w/w of proteins from the bacterial strain used to produce it, when analyzed by Lowry assay (Example 4). Moreover, after separation from the major part of other bacterial culture medium constituents, the isolated EPS of the present disclosure can be concentrated by, without being limited to, filtration, ultrafiltration, evaporation, spray-drying or freeze-drying, or a combination thereof. Therefore, the isolated EPS of the present disclosure is in the form of a concentrate, a suspension, a powder or a freeze-dried form.

In an embodiment, the isolated EPS comprised in the composition of the present disclosure exhibits at least 1 weight average molecular weight distribution, ranging from 40 to 4000 kDa, from 40 to 2000 kDa, from 40 to 1000 kDa, from 40 to 500 kDa, from 40 to 400 kDa, from 40 to 300 kDa, from 40 to 200 kDa, as determined by size exclusion chromatography (HP-SEC). In an embodiment, said isolated EPS presents at least 2 weight average molecular weight distributions, each of them ranging from 40 to 4000 kDa, from 40 to 2000 kDa, from 40 to 1000 kDa, from 40 to 500 kDa, from 40 to 400 kDa, from 40 to 300 kDa, from 40 to 200 kDa, as determined by size exclusion chromatography (HP-SEC).

In an embodiment, the isolated EPS of the present disclosure includes neutral glycosyl units, amino glycosyl units and acid glycosyl units. Examples of neutral glycosyl units include, but are not limited to glucose, rhamnose, mannose, xylose and galactose.

Examples of amino glycosyl units include, but are not limited to galactosamine, glucosamine, N-acetyl glucosamine and N-acetyl galactosamine. Examples of acidic glycosyl units include, but are not limited to uronic acids, including glucuronic acid, galacturonic acid and hexuronic acid. In one embodiment, the isolated EPS of the present disclosure includes neutral glycosyl units comprising glucose, rhamnose, mannose, xylose and galactose. In one embodiment, the isolated EPS of the present disclosure includes amino glycosyl units comprising N-acetyl glucosamine and N-acetyl galactosamine. In one embodiment, the isolated EPS of the present disclosure may include acidic glycosyl units comprising glucuronic acid. In one embodiment, the isolated EPS of the present disclosure comprises between 30 and 90%, between 35 and 80%, between 40 and 75% of neutral glycosyl units with respect to the total number of glycosyl units of said EPS. In one embodiment, the isolated EPS of the present disclosure comprises between 10 and 70%, between 15 and 65%, between 20 and 60% of amino glycosyl units with respect to the total number of glycosyl units of said EPS. In one embodiment, the isolated EPS of the present disclosure comprises between 0 and 15%, between 0 and 10%, between 0 and 5% of acidic glycosyl units with respect to the total number of glycosyl units of said EPS. In another embodiment, the isolated EPS of the present disclosure is structurally composed of glycosyl units comprising mannose, galactose, glucose, N-acetyl galactosamine and N-acetyl glucosamine. In another embodiment, the isolated EPS of the present disclosure is not structurally composed of ribose, arabinose, rhamnose, fructose and/or fucose. In another embodiment, the isolated EPS of the present disclosure is substantially free of ribose, arabinose, rhamnose, fructose and/or fucose.

The isolated EPS of the present disclosure present a strong emulsifying activity even at low concentration (i.e., an E24 of 50% was reached using 50 μg·mL⁻¹ only, corresponding to 0.005% EPS).

In one embodiment, the method of the present disclosure comprises the administration of an effective amount of at least one EPS, optionally provided as a composition. The expression “an effective amount of” means a sufficient concentration of isolated EPS to ensure the attenuation of the virulence of a microbial pathogen infection by inhibition or reduction of the colonization by the microbial pathogens on a treated surface, when compared to an untreated surface. In some embodiment, the concentration of the isolated EPS is ranging from 0.00005% to 0.5% w/w based on the total weight of the composition. In some another embodiment, the concentration of the isolated EPS is ranging from 0.0001% to about 0.1% w/w based on the total weight of the composition. In yet another embodiment, the concentration of the isolated EPS is ranging from or 0.0005% to about 0.08% w/w based on the total weight of the composition.

In another embodiment, the composition of the present disclosure comprising at least 0.000005% w/w w/w of the composition, at least 0.00001% w/w of the composition, at least 0.00002% w/w w/w of the composition, at least 0.00005% w/w of the composition, at least 0.0001% w/w w/w of the composition, at least 0.0002% w/w of the composition, at least 5 0.0005% w/w w/w of the composition, at least 0.001% w/wof the composition, at least 0.002 w/w of the composition, at least 0.004% w/w of the composition, at least 0.005% w/w of the composition, at least 0.0075% w/w of the composition, at least 0.01% w/w of the composition, at least 0.015% w/w of the composition, at least 0.02% w/w of the composition, of at least one isolated EPS having the ability of attenuating the virulence of a microbial pathogen infection by inhibiting or reducing colonization by said microbial pathogens on a treated surface.

In another embodiment, the composition of the present disclosure can be an aqueous solution or suspension comprising from about 0.5-5000 μg·mL⁻¹, from about 1-1000 μg·mL⁻¹, from about 5-500 μg·mL⁻¹ of at least one isolated EPS having the ability of attenuating the virulence of a microbial pathogen infection by inhibiting or reducing colonization by said microbial pathogens onto a treated surface.

In another embodiment, the composition of the present disclosure can be an aqueous solution or suspension comprising at least 50 ng·mL⁻¹, at least 100 ng·mL⁻¹, at least 200 ng·mL⁻¹, at least 500 ng·mL⁻¹, at least 1 μ·mL⁻¹, at least 2 μg·mL⁻¹, at least 5 μg·mL⁻¹, at least 10 μg·mL⁻¹, at least 20 μg·mL⁻¹, at least 40 μg·mL⁻¹, at least 50 μg.·L⁻¹, at least 75 μg·mL⁻¹, at least 100 μg·mL⁻¹, at least 150 μg·mL⁻¹, at least 200 μg·mL⁻¹ of at least one isolated EPS having the ability of attenuating the virulence of a microbial pathogen infection by inhibiting or reducing colonization by said microbial pathogens on a treated surface.

The composition disclosed herein comprises at least one isolated exopolysaccharide originating from a marine bacteria and a suitable carrier. The choice of carrier(s) will be made according to the type of final formulation. In an embodiment, the carrier of the composition herein can increase the biological activity (i.e., antibiofilm and/or antiviral activity) of the composition, but not decrease it. In another embodiment, the carrier of the composition herein is immunologically inert. In another embodiment, the carrier of the composition herein can be a combination of one or more carriers.

In one embodiment, the composition disclosed herein may further comprise another EPS. In one another embodiment, the composition disclosed herein may further comprise polyglutamic acid.

In one embodiment, apart from its antibiofilm activity (for example, in vitro), the isolated EPS as described herein, optionally provided as a composition, do not negatively impact pathogenic bacteria growth, and more generally, the presence of EPS does not influence the growth rate of all bacterial strains tested. In another embodiment, the isolated EPS as described herein, optionally provided as a composition, do not have any bactericidal, bacteriostatic nor antibiotic effect, independently from its concentration and from the pathogenic bacteria considered. In another embodiment, the isolated EPS as described herein, optionally provided as a composition, also respects the integrity of the natural microbiota that is present on biological tissues. In another embodiment, the isolated EPS as described herein, optionally provided as a composition, do not induce cytotoxicity in the subject intended to receive them, regardless of its concentration and its incubation duration.

In one another embodiment, the isolated EPS as described herein, optionally provided as a composition, substantially lacks the ability to induce an immune (cellular and/or humoral) response. In some embodiment, the isolated EPS as described herein, optionally provided as a composition, lacks the ability to induce any immunological response, such as inflammatory response and/or anergy. In another embodiment, the isolated EPS as described herein, optionally provided as a composition, lacks any detectable effect on dendritic cells maturation into immunostimulatory antigen-presenting cells. In yet another embodiment, the isolated EPS as described herein, optionally provided as a composition, fails to induce CD83, CD40 and/or CD25 expression from dendritic cells. In another embodiment, the isolated EPS as described herein, optionally provided as a composition, fails to induce the proliferation of cells expressing IL-6 and/or IL-8. In some another embodiment, the isolated EPS as described herein, optionally provided as a composition, fails to induce the production of inflammatory mediators, such as cytokines, chemokines and/or prostaglandins. In yet another embodiment, the isolated EPS as described herein, optionally provided as a composition, fails to induce the production of IL-6 and/or IL-8.

### Antibiofilm Activity

In the case of bacterial pathogens, pathogenic biofilms can develop on different mucosa and epithelial cells, oral, nasal, pulmonary, digestive, cutaneous, ophthalmic, and urogenital but also on abiotic and hydrophobic non-polar surfaces (e.g., Teflon, glass and plastic) such as central venous, urinary and peritoneal dialysis catheters, endotracheal tubes, pacemakers, joints prosthetics and mechanical heart valves.

Microbial pathogens embedded in such matrix can survive by developing their own ecosystem where they protect themselves from metabolites or substances generated by body's immune defenses and also exogenous treatments while acquiring new abilities, via cell-to-cell communication (e.g., quorum sensing), such as resistance genes in order to adapt and face their environment. Hence, clinically, biofilm formation is known to be a key factor in the establishment and persistence of several difficulties to treat infections. For example, exopolysaccharides production from pathogenic bacteria protect them and make them recalcitrant to antimicrobial treatment.

Therefore, the present disclosure, which aims to attenuate the virulence of a bacterial infection, is not only directed to active or established bacterial infections but also to inhibition of the initial onset of pathogenesis leading to a bacterial infection, namely the early biofilm formation wherein bacteria reversibly adhere on a surface.

In one embodiment, the composition of the present disclosure imparts a biofilm formation inhibitory effect on various bacterial pathogens incubated in optimal conditions on a synthetic surface when compared to untreated surface. In another embodiment, a surface pre-treatment with the EPS in accordance with the present disclosure inhibits early biofilm formation, even when applied at low concentration, when compared to untreated surface. In another embodiment, use of the composition of the present disclosure imparts a reduction of biofilm formation from various bacterial pathogens incubated in optimal conditions on a synthetic surface. In another embodiment, a surface pre-treatment with the composition reduces early biofilm formation, even when applied at low concentration when compared to untreated surface. In another embodiment, the composition of the present disclosure decreases of at least 1%, at least 2%, at least 3%, at least 4%, at least 5%, at least 6%, at least 7%, at least 8%, at least 9%, at least 10%, at least 11%, at least 12%, at least 13%, at least 14%, at least 15% pathogenic bacteria biofilm formation on synthetic surface, when compared to untreated synthetic surface. In another embodiment, the composition of the present disclosure having an EPS concentration of at least 200 μg/mL decreases of at least 5%, at least 6%, at least 7%, at least 8%, at least 9%, at least 10% pathogenic bacteria biofilm formation on synthetic surface, when compared to untreated synthetic surface. In another embodiment, the composition of the present disclosure having an EPS concentration of 400 μg/mL decreases of at least 15%, at least 16%, at least 17%, at least 18%, at least 19%, at least 20% pathogenic bacteria biofilm formation on synthetic surface, when compared to untreated synthetic surface.

In one embodiment, the EPS of the present disclosure has the capacity to reduce and/or limit the consequences of bacterial biofilm installation, regardless its origin, on biological surfaces. Indeed, in addition to the demonstrated prophylactic effect reducing and/or preventing the formation of biofilm, surface washing with compositions in accordance with the present disclosure can also have a therapeutic activity due to its ability to remove pathogenic bacterial cells from a biofilm. In another embodiment, the compositions in accordance with the present description promote biofilm disruption or dissolution (reducing pre-existing colonization and pre-formed or accumulated biofilm) on a surface. In some embodiment, the compositions in accordance with the present description promote early biofilm disruption or dissolution on a surface. In another embodiment, surface treatment with compositions in accordance with the present disclosure prevent, reduce and/or inhibit the recolonization of bacteria on a surface. In another embodiment, the EPS composition of the present disclosure decreases of at least 1%, at least 2%, at least 3%, at least 4%, at least 5%, at least 6%, at least 7%, at least 8%, at least 9%, at least 10% pathogenic bacteria biofilm formation on biological surface, when compared to untreated biological surface. In another embodiment, the composition of the present disclosure having an EPS concentration of at least 200 pg/mL decreases of at least 10%, at least 11%, at least 12%, at least 13%, at least 14%, at least 15%, at least 16%, at least 17%, at least 18%, at least 19%, at least 20% pathogenic bacteria biofilm formation on biological surface, when compared to untreated biological surface. In another embodiment, the composition of the present disclosure having an EPS concentration of 400 pg/mL decreases of at least 15%, at least 16, at least 17%, at least 18%, at least 19%, at least 20%, at least 21%, at least 22%, at least 23%, at least 24%, at least 25%, at least 26%, at least 27%, at least 28%, at least 29%, at least 30% pathogenic bacteria biofilm formation on biological surface, when compared to biological surface.

In another embodiment, surface treatment with compositions in accordance with the present disclosure takes place no later than 8 h, no later than 6 h, no later than 4 h, no later than 3 h, no later than 2 h, no later than 1 h after contact with pathogenic bacteria. The surface treatment with compositions in accordance with the present disclosure can also be applied immediately after contact with pathogenic bacteria. The surface treatment with compositions in accordance with the present disclosure can also be applied as a preventive measure before any contact with pathogenic bacteria. The surface treatment with compositions in accordance with the present disclosure can thus be applied at least min, at least 10 min, at least 15 min, at least 30 min, at least 60 min, at least 2 h, at least 3 h, at least 4 h, at least 6 h, at least 8 h before potential contact with pathogenic bacteria.

### Antiviral Activity

Viral diseases or infections are transmitted by viruses that attack the body at one or more specific points. For example, an individual can contract a viral disease through direct contact with another individual who is already sick, i.e., through biological fluids or aerosols as well as with contact of infected surfaces.

Once inoculated into the body, the virus enters specific host cells, diverts the cellular machinery to multiply and the virions synthesized this way will in turn spread and infect adjacent cells.

The present disclosure is also directed to attenuating the virulence of a viral infection by inhibiting and/or reducing the colonization of viral pathogens on a treated surface. In other words, viral pathogens surface colonization reduction and/or inhibition is achieved by reducing living cells mortality compared to untreated cells and/or by preventing the release of virions as well as their spreading into adjacent living cells.

In one embodiment, pre-treatment of living cells with compositions in accordance with the present disclosure prior to virus inoculation, highlights the prophylactic character of EPS compositions against viral infections, regardless the virus considered. In an embodiment, pre-treatment of living cells with compositions in accordance with the present disclosure prior to virus inoculation reduces the cell's mortality vs. untreated cells. In yet another embodiment, pre-treatment of living cells (i.e., prior to virus inoculation of the living cells) with compositions in accordance with the present disclosure is administered in a single dose before viral inoculation. In another embodiment, pre-treatment of living cells with compositions in accordance with the present disclosure prior to virus inoculation, increases cells survival after viral infection (regardless the virus considered), when compared to untreated cells survival. In another embodiment, pre-treatment of living cells with compositions in accordance with the present disclosure prior to virus inoculation (1 TCID50), increases cells survival rate of at least 10%, at least 11%, at least 12%, at least 13%, at least 14%, at least 15%, when compared to untreated-cells survival rate. In another embodiment, pre-treatment of living cells with compositions in accordance with the present disclosure prior to virus inoculation (0.001 TCID50), increases cells survival rate of at least 15%, at least 16%, at least 17%, at least 18%, at least 19%, at least 20%, when compared to untreated-cells survival rate.

In one embodiment, post-treatment of living cells (i.e., after virus inoculation of the living cells) with compositions in accordance with the present disclosure, prevents the release of virions as well as their spreading into adjacent cells. In another embodiment, post-treatment of living cells (i.e., after virus inoculation of the living cells) with compositions in accordance with the present disclosure, provides a therapeutic effect that reduces the virulence of a viral infection vs. untreated cells, for at least 1 day, for at least 2 days, for at least 3 days, for at least 4 days, at least 5 days of viral inoculation. In yet another embodiment, post-treatment of living cells (i.e., after virus inoculation of the living cells) with compositions in accordance with the present disclosure is administered in a single dose after viral inoculation. In another embodiment, without wishing to be bound by theory, the administration of the EPS compositions in accordance with the present disclosure on human living cells creates an extracellular “barrier” that sterically hinders the internalization of viruses, regardless the virus considered. In an embodiment, post-treatment of living cells with EPS compositions in accordance with the present disclosure, after virus inoculation, increases cells survival rate when compared to untreated cells survival rate. In another embodiment, post-treatment of living cells with EPS compositions in accordance with the present disclosure, after virus inoculation (1 TCID50), increases cells survival rate of at least 1%, at least 2%, at least 3%, at least 4%, when compared to untreated-cells survival rate. In some another embodiment, post-treatment of living cells with compositions in accordance with the present disclosure after virus inoculation (0.001 TCID50), increases cell survival rate of at least 25%, at least 26%, at least 27%, at least 28%, at least 29%, at least 30%, when compared to untreated-cells survival rate.

In another embodiment, surface treatment with compositions in accordance with the present disclosure prevent, reduce and/or inhibit the recolonization of viruses on a surface.

In another embodiment, surface treatment with compositions in accordance with the present disclosure takes place no later than 8 h, no later than 6 h, no later than 4 h, no later than 3 h, no later than 2 h, no later than 1 h after contact with a virus. The surface treatment with EPS compositions in accordance with the present disclosure can also be applied immediately after contact with a virus. The surface treatment with compositions in accordance with the present disclosure can also be applied as a preventive measure before any contact with a virus. The surface treatment with compositions in accordance with the present disclosure can thus be applied at least 5 min, at least 10 min, at least 15 min, at least 30 min, at least 60 min, at least 2h, at least 3h, at least 4h, at least 6h, at least 8 h before potential contact with a virus.

### Pathogens

The compositions in accordance with the present disclosure are effective for attenuating the virulence of a microbial pathogen infection by inhibiting or reducing colonization of microbial organisms, such as bacteria, fungi, yeasts and viruses onto surfaces treated with the claimed composition.

In an embodiment, compositions in accordance with the present disclosure are effective for attenuating the virulence of a bacterial pathogen infection by inhibiting or reducing colonization of bacteria. In some embodiment, bacteria for which the disclosure may be relevant for the dedicated usage include both gram-negative and gram-positive bacteria such as, without being limited to bacteria belonging to Staphylococcus species, Streptococcus species, Enterococcaceae and Enterococcus species, Neisseria or Branhamella species, Bacillus species, Propionibacterium species, Corynebacterium species, Listeria species, Clostridium species, Escherichia species, Enterobacter species, Proteus species, Pseudomonas species, Klebsiella species, Salmonella species, Shigella species, Campylobacter species, Actinomyces species, Actinobacillus species, Acinetobacter species, Aggregatibacter species, Fusobacterium species, Haemophilus species, Mycobacterium species, Pseudomonas species, Porphyromonas species, Bacteriodes species, Treponema species, Prevotella species, or Eubacterium species. In some another embodiment, bacteria for which the disclosure may be relevant for the dedicated usage include both gram-negative and gram-positive bacteria such as, without being limited to Staphylococcus aureus, Methicillin Resistant Staphylococcus aureus (MRSA), Streptococcus pyogenes (group A), Streptococcus spp. (viridans group), Streptococcus agalactiae (group B), Streptococcus bovis, Streptococcus (anaerobic species), Streptococcus pneumoniae, Streptococcus mutans, S. sanguis, S. oralis, S. mitis, S. salivarius, S. gordonii, Neisseria gonorrhoeae, Neisseria meningitidis, Branhamella catarrhalis, Bacillus anthracis, Bacillus subtilis, Propionibacterium acnes, Corynebacterium diphtheriae, Listeria monocytogenes, Clostridium tetani, Clostridium difficile, Escherichia coli, Proteus mirablis, Pseudomonas aeruginosa, Klebsiella pneumoniae, Campylobacter jejuni, Actinobacillus actinomycetumcomitans, Acinetobacter baumannii, Aggregatibacter actinomycetemcomitans, Fusobacterium nucleatum, Haemophilus influenzae, Mycobacterium tuberculosis, Pseudomonas aeruginosa, Porphyromonas gingivalis, Bacteriodes forcythus, Treponema denticola, Prevotella intermedia, or Eubacterium nodatum. In an embodiment, the bacteria species is Pseudomonas aeruginosa, Staphylococcus aureus, Klebsiella pneumoniae, Haemophilus influenzae, Streptococcus pneumoniae, Streptococcus mutans, Moraxella catharalis, Propionibacterium acnes (also known as Cutibacterium acnes) or a combination thereof. In yet another embodiment, the bacteria species are Staphylococcus aureus, Haemophilus influenzae, Streptococcus pneumoniae, Moraxella catharalis, Propionibacterium acnes (also known as Cutibacterium acnes), or a combination thereof.

Thus, compositions in accordance with the present disclosure may be useful for attenuating the virulence of a bacterial pathogen infection by inhibiting and/or reducing colonization on treated surfaces to prevent, attenuate microbial infection(s), and/or reduce the likelihood of microbial infection(s), including, without being limited to: bacteremia, septicemia, pneumonia, meningitis, osteomyelitis, endocarditis, dental caries, periodontal disease, sinusitis, rhinitis, pink eye, urinary tract infections, tetanus, gangrene, colitis, acute gastroenteritis, impetigo, acne, acne rosacea, red sore, atopic dermatitis, psoriasis, wound infections, burn infections, fasciitis, bronchitis, or a variety of abscesses, nosocomial infections, and/or opportunistic infections.

In another embodiment, the compositions in accordance with the present disclosure are effective for attenuating the virulence of a viral pathogen infection by inhibiting or reducing colonization of viruses on a surface or on an epithelium. EPS compositions in accordance with the present disclosure are effective against certain viruses, such as HIV, herpes simplex virus, cytomegalovirus and/or human papillomavirus but also against rhinovirus, orthomyxovirus, paramyxovirus, coronavirus, adenovirus, influenza, Rouse Sacorma virus, parainfluenza, metapneumovirus and/or Epstein-Barr virus.

Thus, the compositions in accordance with the present disclosure are also effective for preventing and/or treating infections such as cold, flu, cold sores, genital herpes, warts and/or effective for preventing HIV and SARS-CoV-2 infections. In some embodiment, the viruses are Influenza, Coronavirus, SARs-CoV-2, Parainfluenza, Respiratory Syncytial Virus, Metapneumovirus, or a combination thereof.

### Biological Surfaces Applications

In one embodiment, the compositions and methods in accordance with the present disclosure are applicable to human or more generally to animal tissues comprising, without being limited to: cells, epithelial cells and/or mucosa. The considered biological surfaces in the present disclosure are, without being limited to: the oral cavity, the dental surface, the nasal cavity, the respiratory tract, the throat, the ears, the ophthalmic region, the urogenital tract, the skin, the scalp, the hairs and/or the nails. In some embodiment, the way of administration of the compositions in accordance with the present disclosure is nasal, respiratory and/or buccal routes.

In an embodiment, the compositions and methods in accordance with the present disclosure can be administered to healthy subjects having intact and healthy tissues but are useful when the subjects have disrupted tissues following wound or scratch that may lead to local and/or systemic infections. In another embodiment, the compositions and methods in accordance with the present disclosure can be administered to subjects susceptible of being infected. In another embodiment, the compositions and methods in accordance with the present disclosure can be administered to infected subjects.

In an embodiment, the subjects administered with the composition and methods in accordance with the present disclosure are animals. In another embodiment, the subjects administered with the composition and methods in accordance with the present disclosure are mammals. In yet another embodiment, the subjects administered with the composition and methods in accordance with the present disclosure are humans.

In an embodiment, for intra-nasal administration, the compositions in accordance with the present disclosure can be administered in the form of ointments or gels to be applied directly on the nasal mucosa. In another embodiment, the compositions in accordance with the present disclosure can be administered via nose drops, a liquid spray, such as via a plastic bottle atomizer, bag-on-valve aerosol and/or metered-dose inhaler. Nasal solutions are usually aqueous solutions designed to be administered to the nasal passages in drops or sprays and are prepared so that they are similar in many respects to nasal secretions, so that normal ciliary action can be maintained. In one embodiment, the aqueous nasal solutions are usually isotonic or may be hypertonic, presenting final saline concentrations between 0.1% to 10% w/w based on the total weight of the nasal composition. In some embodiments, the aqueous nasal solutions are usually isotonic or may be hypertonic, presenting final saline concentrations between 0.5% to 5% w/w based on the total weight of the nasal composition. In some another embodiment, the aqueous nasal solutions are usually isotonic or may be hypertonic, presenting final saline concentrations between 0.7% to 3% w/w based on the total weight of the nasal composition.

For respiratory administration, also known as inhalations, nebulization or insufflations, the compositions in accordance with the present disclosure are administered via pressurized aerosols as a fine powder suspension or as a solution, in adjunction with a liquefied gas propellant. When released through a suitable valve and oral adapter, the composition is propelled into the respiratory tract of the subject. Fine mists are produced by pressurized aerosols (i.e., insufflator, nebulizer) and hence their use in considered advantageous. Pressurized aerosols may comprise a suitable propellant such as dichlorodifluoromethane, trichlorofluoromethane, dichlorotetrafluoroethane, nitrogen, carbon dioxide or other suitable gas. In the case of a pressurized aerosol, the dosage unit may be determined by providing a valve to deliver a metered amount.

The compositions in accordance with the present disclosure may be topically applied to the mucosal tissue of the oral cavity, to the gingival tissue of the oral cavity, and/or to the surface of the teeth in several conventional ways. For example, the gingival or mucosal tissue may be rinsed with a solution (e.g., mouth rinse, mouth spray) containing the compositions in accordance with the present disclosure; or if the compositions in accordance with the present disclosure is in the form of a dentifrice (e.g., toothpaste, tooth gel or tooth powder), the gingival/mucosal tissue or teeth is bathed in the liquid and/or lather generated by brushing the teeth. Other non-limiting examples include applying a non-abrasive gel or paste containing the claimed composition, directly to the gingival/mucosal tissue or to the teeth with or without an oral care appliance described below; chewing gum that contains the claimed composition; chewing or sucking on a breath tablet, lozenge or dissolvable strip which contains the claimed composition. In some embodiment, the methods of applying the composition to the gingival/mucosal tissue and/or the teeth are via rinsing with a mouth rinse solution or via brushing with a dentifrice. In some another embodiment, the methods of applying the compositions in accordance with the present disclosure to the gingival/mucosal tissue and/or the teeth are via plastic bottle atomizer or bag-on-valve aerosol. Other methods of topically applying EPS compositions in accordance with the present disclosure to the gingival/mucosal tissue and the surfaces of the teeth are apparent to those skilled in the art.

For vaginal administration, the compositions in accordance with the present disclosure may be presented as pessaries, tampons, suppositories, creams, gels, pastes, foams or sprays containing in addition to the active ingredient such carriers as are known in the art to be appropriate.

When formulated for topical administration, the compositions in accordance with the present disclosure may contain ingredients typical in topical pharmaceutical, medical device such as wound dressings or cosmetic compositions, such as a carrier, vehicle or medium. Specifically, the carrier, vehicle, or medium is compatible with the tissues to which it will be applied, such as the oral cavity, the dental surface, the nasal cavity, the respiratory tract, the throat, the ears, the ophthalmic region, the urogenital tract, the skin, the scalp, the hairs, the nails or mucosa. The compositions and components of the disclosure are suitable for contacting infected tissues or for use in subjects in general without undue toxicity, incompatibility, instability, allergic response, and the like. As appropriate, EPS compositions in accordance with the present disclosure may comprise any additional ingredient conventionally used in the fields under consideration.

In terms of their form, the compositions in accordance with the present disclosure may include solutions, emulsions (including microemulsions), suspensions, creams, lotions, gels, powders, or other typical solid or liquid compositions used for application to skin and other tissues where the compositions may be used. Such compositions may contain: antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents or water, and optionally including anesthetics, anti-itch actives, botanical extracts, conditioning agents, darkening or lightening agents, glitter, humectants, mica, minerals, polyphenols, silicones or derivatives thereof, sunblocks, vitamins, or phytomedicinals. In still another embodiment, EPS compositions in accordance with the present disclosure are formulated with the above-mentioned ingredients providing the EPS compositions with a long-term stability, which may be needed for a continued or long-term treatment.

In term of way of administration, the carrier of the compositions in accordance with the present disclosure may be selected to provide residence time of the composition on the treated surface of at least 1 minute, or at least 5 minutes, or at least 10 minutes, or at least 15 minutes, or at least 20 minutes, or at least 25 minutes, or at least 30 minutes, or at least 1 h, or at least 2 h, or at least 4 h, or at least 8 h following application.

In the case of liquid formulations, the volume of the claimed composition can be adjusted according to the estimation of the overall surface area of the tissue to be treated, for a given human or animal. For example, about 1 mL to about 30 mL, such as, for example, about 3 mL to about 25 mL, or about 5 mL to about 20 mL for a mouthwash formulation; or about 0.05 mL to about 2 mL, such as about 0.1 mL to about 1.5 mL for a nasal spray formulation. In some embodiment, the volume of the claimed composition dedicated to nasal formulations is about 50 μL to about 300 μL per nostril, or about 100 μL to 200 μL per nostril for bag-on-valve aerosol delivery. In some another embodiment, the volume of the claimed composition dedicated to nasal formulations is about 0.3 mL to 2 mL, or about 0.6 mL to 1.2 mL per nostril for plastic bottle atomizer delivery. Other delivery media, such as dissolvable strips, may have dosages derived from these ranges given the adjustments for concentrations and other factors known to those of skill in the art.

The daily dosage of the compositions in accordance with the present disclosure typically depends on the subject condition (healthy for a prophylactic treatment or sick for a curative treatment) and also on the surface to be treated. The daily dosage may be once a day or divided into two or more dosages administered two or more times per day.

In an embodiment the compositions in accordance with the present disclosure are administered daily for a period of time being at least 1 day, at least 3 days, at least 5 days, or at least 1 week, or at least 2 weeks, or at least 3 weeks, or at least 4 weeks, or at least 1 month, or at least 2 months, or at least 3 months. In one non-limiting embodiment it may be desired that the treatment is continued until no pathogenic bacteria are detectable on the treated surface. In another embodiment EPS compositions in accordance with the present disclosure are administered to a subject as a prophylactic treatment, such as once a day or twice a day for an undetermined period.

The compositions in accordance with the present disclosure can be used alone or in combination with another antibiotic/antimicrobial/anti-viral agent. For example, the inhibition of biofilm production would make a microbial pathogen much more susceptible to the action of another antibiotic/antimicrobial/anti-viral agent, such as those conventionally used to treat microbial pathogen-specifics. Similarly, the compositions in accordance with the present disclosure can also be used alone or in combination with another EPS.

### Non-Biological Surface Applications

In an embodiment, the compositions in accordance with the present disclosure can also be applied on non-biological surfaces comprising without being limited to hydrophobic and non-polar surfaces. Essentially any medical device which is susceptible of being colonized by pathogenic microorganisms and/or coated at least partially by a biofilm is appropriate for the practice of the present disclosure, including contact lenses, contact lens cases, ophthalmic and lens treatment solution containers, or other related products, analyte sensing devices such as electrochemical glucose sensors, drug delivery devices such as insulin pumps, devices which augment hearing such as cochlear implants, urine contacting devices (for example, urethral stents, urinary catheters), blood contacting devices (including cardiovascular stents, venous access devices, valves, vascular grafts, hemodialysis and biliary stents), or body tissue and tissue fluid contacting devices (including biosensors, implants and artificial organs). Medical devices that could be treated with the compositions include but are not limited to permanent catheters, (e.g., central venous catheters, dialysis catheters, long-term tunneled central venous catheters, short-term central venous catheters, peripherally inserted central catheters, peripheral venous catheters, pulmonary artery Swan-Ganz catheters, urinary catheters, or peritoneal catheters), long-term urinary devices, tissue bonding urinary devices, vascular grafts, vascular catheter ports, wound drain tubes, ventricular catheters, hydrocephalus shunts, cerebral or spinal shunts, heart valves, heart assist devices (e.g., left ventricular assist devices), pacemaker capsules, incontinence devices, penile implants, small or temporary joint replacements, urinary dilator, cannulae, elastomers, hydrogels, surgical instruments, dental instruments, tubings, such as intravenous tubes, breathing tubes, dental water lines, dental drain tubes, or feeding tubes, fabrics, paper, indicator strips (e.g., paper indicator strips or plastic indicator strips), adhesives (e.g., hydrogel adhesives, hot-melt adhesives, or solvent-based adhesives), bandages, wound dressings, orthopedic implants, or any other device used in the medical field. Additional medical devices also include, but are not limited to, any device which may be inserted or implanted into a human being or other animal, or placed at the insertion or implantation site such as the skin near the insertion or implantation site, and which include at least one surface which is susceptible to colonization by biofilm embedded microorganisms. Medical devices also include any other surface which may be desired or necessary to prevent biofilm embedded microorganisms from growing or proliferating on at least one surface of the medical device, or to remove or clean biofilm embedded microorganisms from the at least one surface of the medical device, such as the surfaces of equipment in operating rooms, emergency rooms, hospital rooms, clinics, or bathrooms.

The compositions in accordance with the present disclosure can be administered with the adjunction of an acceptable carrier onto a decontaminated non-biological surface that will be in contact with a biological surface. In some instances, the functionality or physical stability of the compositions in accordance with the present disclosure can also be increased by the addition of various additives to aqueous solutions or suspensions. Additives, such as, without being limited to polyols (including sugars), amino acids, surfactants, polymers, other proteins or certain salts may be used. The compositions and method in accordance with the present disclosure can be used alone or in combination with another disinfection treatment. For example, the inhibition of biofilm production would make a microbial pathogen much more susceptible to the action of another disinfection treatment, such as those conventionally used to kill or discard microbial pathogen-specifics from treated surface.

In an embodiment, the compositions in accordance with the present disclosure are applied, dipped and/or coated on a medical device or apparatus prior to the medical device or apparatus encountering a contaminated environment and/or prior to the object or apparatus encountering the tissues of a subject. In another embodiment, both the tissues of the subject and the medical device or apparatus in contact with said tissues can be treated with the compositions in accordance with the present disclosure in order to limit pathogen adhesion and/or to reduce the risk of infections (once in contact with the tissue of the subject). In another embodiment, the compositions in accordance with the present disclosure may be used to facilitate the decontamination process of reusable medical material or equipment. In some further embodiment, the compositions in accordance with the present disclosure may be used to facilitate the decontamination process of reusable medical material or equipment, before and/or after the medical material or equipment sterilization.

The surface treated with the compositions in accordance with the present disclosure, by spraying or soaking, needs to be partially covered or fully covered in order to reduce pathogen adhesion onto said surface. The applied EPS compositions in accordance with the present disclosure can be effective to cover a substantial percentage of the treated surface, such as, for example, at least 10%, at least 20%, at least 30%, at least 40%, at least 50%, at least 60%, at least 70%, at least 80%, at least 90% of the overall surface.

### EXAMPLES

### Example 1: EPS Production Process

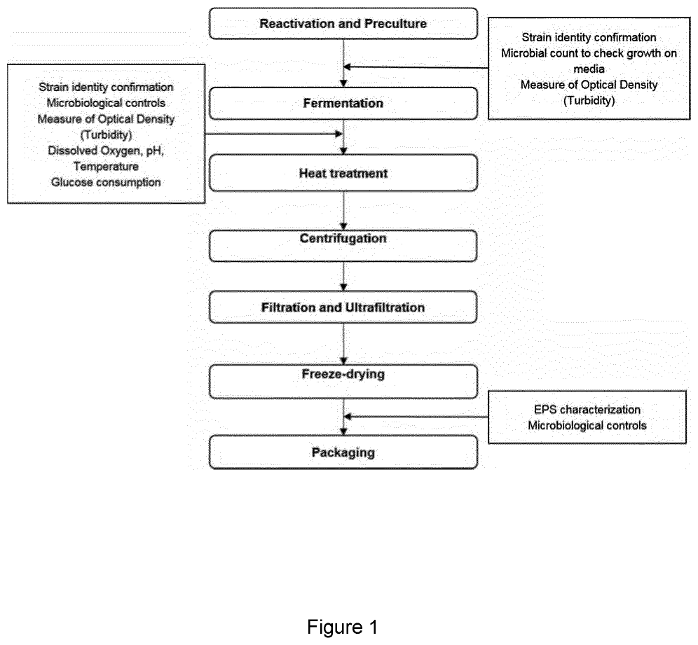

A 7-step process implemented for EPS production is depicted in FIG. 1. Briefly:

Reactivation and Preculture: The content of a cryotube containing Bacillus licheniformis LP-T14 (deposit number NCIMB 43557) was separated in pre-culture tubes and then suspended in 9 mL of adapted liquid Zobell medium, respecting sterile conditions. The pre-culture tubes were then placed under orbital agitation at 40° C. and were used to seed liquid medium test made in 150 mL Erlens under agitation for 5 h at 40° C. in liquid Zobell medium. Then, 4 fermenters of 5 L were seeded by the 150 mL Erlen pre-culture and were placed for 4-8 h at 40° C., under controlled agitation, oxygenation (30-60%) and pH (5-8) until optical density (OD) >2 to ensure that the bacterial growth is at the beginning of the exponential phase.

Fermentation: A 500 L fermenter was filled with 150 L sterile fermentation media composed of sea salts (40 g/L), tryptone (6 g/L), yeast extract (6 g/L), antifoam (0.33 mL/L), dextrose (12 g/L) and deionized H₂O (qsp.). The fermentation culture medium was inoculated with 5 L fermentation pre-cultures and placed for 20-40 h at 40° C., under controlled agitation, an oxygenation of 30-60 % and pH 5-8. Both the strain growth (OD) and glucose consumption were monitored until the end of the fermentation.

Heat treatment: Heat treatment was performed on 400 L jacketed stirred tank for 1 hour at 85° C. to inactivate enzymes and bacteria.

Centrifugation: The whole culture medium was centrifuged using disk stack centrifuge, 14000 g, flow 200-800 L/h for biomass and supernatant separation.

Filtration and Ultrafiltration: The supernatant was further processed by successive filtration (1.60-0.22 μm) and ultrafiltration steps (10-100 kDa cut-off cartridges hollow fiber PolySulfone) in order to purify and recover only high molecular weight compounds; i.e., EPS.

Freeze-drying: EPS were frozen at −20° C. for at least 16 hours, then freeze-dried in a 100 kg ice capacity Freeze-dryer.

Packaging: Freeze-dried EPS were packaged in low density polyethylene bag sealed under vacuum and labelled, prior to being formulated into appropriate formulation. Samples of each batch were characterized according to standard procedures.

### Example 2: EPS Characterization—Average Molecular Mass Determination

The EPS used in the example herein was obtained by fermentation of Bacillus licheniformis LP-T14 (deposit number NCIMB 43557) as described in Example 1.

The EPS were analyzed by a size exclusion chromatography (HP-SEC) apparatus, namely TDA 302 (Viscotek), equipped with Refractive Index and Laser Light Scattering and Viscometers detectors. The system was calibrated with a Pullulan standard, with certified molecular weight, polydispersity index and intrinsic viscosity (PolyCAL-PullulanSTD-105k, Malvern Panalytical). EPS samples (100 μL, 1 mg/mL) dissolved in mobile phase (0.2 M NaNO₃+0.05% NaN₃, pH 6.81) were injected in a GMPWX column (7.8 mm×30 cm, Tosoh Bioscience) at 40° C. under a flow rate of 0.6 mL/min.

The analysis results (average values of two analyses per EPS sample) are reported in terms of weight-average molecular weight (Mw; 40-150 kDa), number-average molecular weight (Mn; 26-100 kDa), and polydispersity index (Mw/Mn; 1.2-1.8), all using a do/dc parameter of 0.135-0.147; and in terms of Recovery % (94-99%), which represents the ratio between the concentration calculated from Refractive Index detector and the experimental concentration.

### Example 3: EPS Characterization—Glycosyl Composition Determination

The EPS used in the example herein was obtained by fermentation of Bacillus licheniformis LP-T14 (deposit number NCIMB 43557) as described in Example 1.

The glycosyl composition of the EPS were performed according to 2 characterization methods, namely the Alditol Acetate Method (Peña et al. (2012) Methods Enzymol. 510:121-39) and the TMS-derivatized methyl glycosides method (Santander et al. (2013) Microbiology 159:1471). Briefly:

Glycosyl Composition Analysis by Alditol Acetate (AA) Method: Glycosyl composition analysis was performed by combined gas chromatography/mass spectrometry (GC/MS) of the alditol acetates. The EPS samples (250 μg) were hydrolyzed in 2 M trifluoroacetic acid (TFA) for 2 h in a sealed tube at 120° C., reduced with NaBD4, and acetylated using acetic anhydride/TFA. The resulting alditol acetates were analyzed on an Agilent 7890A GC interfaced to a 5975C MSD, electron impact ionization mode. Separation was performed on a 30 m Supelco SP-2331 bonded phase fused silica capillary column. Separation of the amino sugars was accomplished using a separate EC-1 column.

Glycosyl Composition Analysis by GC-MS of TMS-Derivatized Methyl Glycosides: Glycosyl composition analysis was performed by combined gas chromatography/mass spectrometry (GC-MS) of the per-O-trimethylsilyl (TMS) derivatives of the monosaccharide methyl glycosides produced from the sample by acidic methanolysis. The EPS samples (250 μg) were heated with methanolic HCl in a sealed screw-top glass test tube for 18 h at 80° C. After cooling and removal of the solvent under a stream of nitrogen, the samples were re-N-acetylated and dried again. The samples were then derivatized with Tri-Sil® (Pierce) at 80° C. for 30 min. GC/MS analysis of the TMS methyl glycosides was performed on an Agilent 7890A GC interfaced to a 5975C MSD, using an Supelco Equity-1 fused silica capillary column (30 m×0.25 mm ID).

The two composition methods show mannose to be the main hexose sugar detected and N-acetyl galactosamine to be the main amino sugar present. There are also significant amounts of galactose, glucose and N-acetyl glucosamine in the samples. Low proportion of xylose has also been detected, whereas glucuronic acid was only detected using the TMS method. However, no trace of ribose, nor arabinose, nor rhamnose, nor fucose, nor fructose were detected using both characterization methods, as presented in Table 1.

TABLE 1
Glycosyl composition analyses (Alditol acetate and TMS methods) of the
EPS produced by fermentation of Bacillus licheniformis LP-T14 (deposit
number NCIMB 43557). Results are expressed in terms of relative
proportion of detected glycosyl units. ND stands for Non-Detected.

| | Alditol acetate Method | TMS method |
|---|---|---|
| Glycosyl residue | (Mol %) | (Mol %) |
| Ribose | ND | ND |
| Arabinose | ND | ND |
| Rhamnose | ND | ND |
| Fucose | ND | ND |
| Fructose | ND | ND |
| Xylose | 1.5 | 1.8 |
| Glucuronic acid | ND | 4.2 |
| Mannose | 23.1 | 35.0 |
| Galactose | 14.2 | 24.7 |
| Glucose | 5.1 | 9.1 |
| N-Acetyl Galactosamine | 46.7 | 18.4 |
| N-Acetyl Glucosamine | 9.4 | 6.8 |
| Total | 100 | 100 |

### Example 4: EPS Protein and Nucleic Acid Content Analysis

The EPS used in the example herein was obtained by fermentation of Bacillus licheniformis LP-T14 (deposit number NCIMB 43557) as described in Example 1.

EPS Protein content analysis: Protein content was determined using a modified Lowry assay, namely the BioRad total protein stain. The analysis was carried out per the manufacturer's instructions on neat EPS samples and quantitated with a BSA standard curve. Briefly, 10 μL of sample was mixed with 200 μL of a 1:4 dilution of the reagent. After minutes, the samples were analyzed by absorbance at 595 nm. Protein contaminants in EPS were evaluated at 0.052±0.019 mg·mL⁻¹, representing around 0.005% w/w based on the total weight of isolated EPS.

EPS nucleic acid content measurements by 260/280 and 260/230 analysis: Two microliters of EPS samples were analyzed on a Nanodrop spectrophotometer at 260/280 and 260/230 nm. Nucleic acid contaminants in EPS was evaluated at 58.0±8.7 ng·μL ⁻¹, representing around 0.006% w/w based on the total weight of isolated EPS.

### Example 5: In Vitro EPS Antibiofilm Activity Against Bacterial Pathogens

The EPS used in the example herein was obtained by fermentation of Bacillus licheniformis LP-T14 (deposit number NCIMB 43557) as described in Example 1.

The following in vitro experiments describe the EPS inhibition effect on pathogenic strains biofilm formation, namely: Gram-negative bacteria [Haemophilus influenzae type b ATCC 9795, Klebsiella pneumoniae ATCC 8047 and Pseudomonas aeruginosa ATCC 27853] and Gram-positive bacteria [Staphylococcus aureus ATCC 29213, Streptococcus pneumoniae serotype 3 ATCC 6303 and Streptococcus mutans DSM 20523].

Bacterial pathogens biofilm formation was carried out in 96-well polystyrene microtiter plates, wherein aliquots of 180 μl of overnight culture (1.5×10⁸ bacteria·mL⁻¹) of each bacterial pathogen were inoculated together with 20 μl of EPS solution in PBS at different concentrations (50, 100, 200 and 400 μg·ml¹) or 20 μl PBS as control. The plates were incubated at 37° C. for 24 h (St. pneumoniae), 48 h (H. influenzae, P. aeruginosa, K. pneumoniae, S. aureus), and 96 h (St. mutans), respecting the growth rate of each strain and without shaking for biofilm production. The incubation media were also adapted to each strain, thus microaerophilic or anaerobic strains incubation was performed in 5% of CO₂, or anaerobic conditions, respectively. Non-adherent bacteria were removed by washing 3 times with distilled sterile water and the adherent bacteria (biofilms) were stained with 0.1% crystal violet solution in ethanol 96% vol (w/v) for 25 min. Excess stain was removed by aspiration, and the plates were washed with distilled sterile water (5 times) and air dried (for 15 min). The stained biofilms were solubilized with ethanol 96% and their optical density (OD) was measured at 585 nm using a microtiter plate reader (Thermo Scientific™ Multiskan™ GO Microplate Spectrophotometer). Biofilm formation (%) was calculated according to the following formula:

$$Biofilm formation ( \% ) = ( 1 - \frac{OD 585 control - OD 585 sample}{OD 585 control} ) \times 100$$

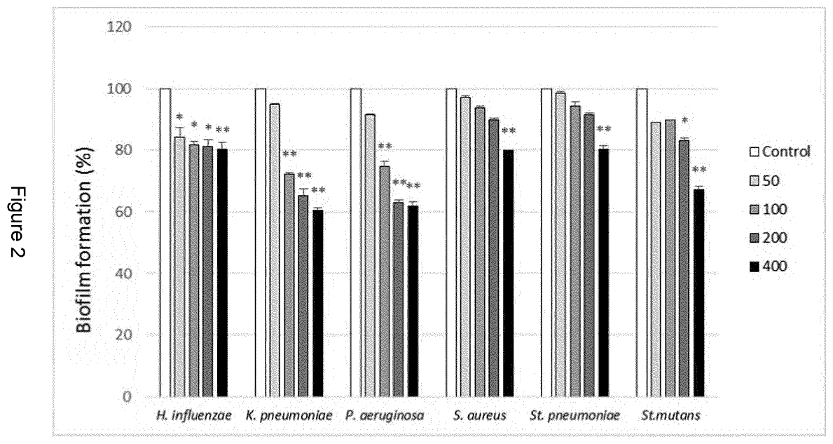

The biofilm inhibitory effect of EPS at different concentrations (from 0 to 400 μg·ml⁻¹) on various bacterial pathogens incubated in optimal conditions is presented in FIG. 2. These experiments were performed in triplicates (n=3) in 2 independent occasions (N=2). Statistical significance was determined by one-way ANOVA and p values were calculated using two-tailed t-test for unequal variance when appropriate. A p value <0.05 was considered as statistically significant.

In these experiments, the pathogenic bacterial strains were cultured and incubated in optimal conditions, in an adapted medium and without competition from other existing pathogens. The percentage of biofilm formed is calculated by OD, so the more crystal violet marked cells are measured after washing, the more cells are attached to the plate and therefore the more biofilm has been formed during the incubation period. A reduction of biofilm formation has been observed, regardless of the pathogenic strain involved. EPS prophylactic antibiofilm action is represented in FIG. 2. Maximal efficiency is observed when the EPS is added within the first 2 hours after inoculation of the pathogenic bacteria (data not shown). When EPS is added 4 hours after inoculation, its effect on biofilm formation is slightly less pronounced (data not shown).

### Example 6: In Vitro EPS Antibacterial Activity Experiments

The EPS used in the example herein was obtained by fermentation of Bacillus licheniformis LP-T14 (deposit number NCIMB 43557) as described in Example 1.

To investigate whether the inhibitory effects of EPS on biofilm formation by bacterial pathogens might be due to direct growth inhibition, antibacterial activity was evaluated performing similar experiments than Example 5 in triplicates (n=3) in 2 independent occasions (N =2). Briefly: Polystyrene microtiter plates were filled with overnight pathogenic bacteria cultures in their respective optimal medium (180 μl) of the tested strain (0D600 =0.1) with 20 μl EPS in PBS solution at a final concentration 50, 100, 200 and 400 μg·ml or with 20 μl of PBS as control. Plates were incubated at 37° C. in the range of time from 24 h to 96 h (optimal growth duration which depends on the strain tested) in aerobic, microaerobic or anaerobic conditions, without shaking. The antibacterial activity was determined measuring the OD600 values using microtiter plate reader (Thermo Scientific™ Multiskan TM GO Microplate Spectrophotometer) and comparing the average OD600 of the control wells to the average OD600 of each tested condition.

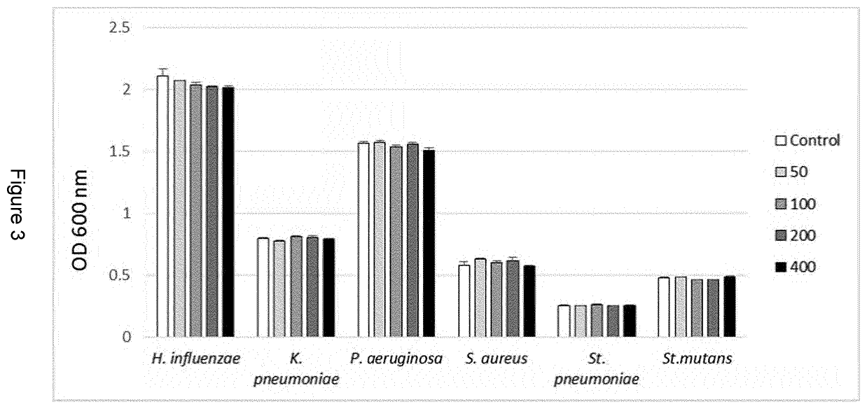

The antibacterial effect of EPS on various bacterial pathogens culture growth is depicted in FIG. 3, comparing OD600 values, mean±SD of treated vs. untreated cells after their respective optimal growth duration. Turbidity measurements of a sample (such as OD600) is commonly used in spectrophotometry for estimating the concentration of bacteria in a medium, allowing to measure the growth over time of a cell population. The growth cultures in absence of EPS were used as control.

The results presented in FIG. 3 show that same pathogenic bacteria growth level was achieved with or without EPS. It demonstrates that the presence of EPS at various concentrations (50-400 μg·ml⁻¹) does not negatively impact pathogenic bacteria growth, and more generally, the presence of EPS did not influence the growth rate of all bacterial strains tested. Therefore, EPS does not have any bactericidal, bacteriostatic nor antibiotic effect, independently from its concentration and from the pathogenic bacteria considered. Thus, it is demonstrated that the presence of EPS, which has no biocidal effect, respects the integrity of the natural microbiota that would be present on biological tissues.

### Example 7: EPS Cytotoxicity Assays

The EPS used in the example herein was obtained by fermentation of Bacillus licheniformis LP-T14 (deposit number NCIMB 43557) as described in Example 1.

Preliminary test of the putative cytotoxic effect of EPS on human nasal epithelial cells (HNEpC) were conducted in vitro. Human Nasal Epithelial Cells (HNEpC) were grown until 80% confluence in BEBM complete medium, as recommended by the manufacturer. Cells were washed twice with PBS and cultured for 2 h or 24 h with EPS ranging from 0 (Control) to 800 μg·mL^{-1 l at} 37° C., 5% CO₂ in medium. After culture, cells were washed twice with

PBS, collected and viability was assessed by staining with viability dye TO-PRO3. Percentage of TO-PRO3 negative cells, representing viable cells in the culture, were evaluated by Symphony BD Sciences flow cytometer.

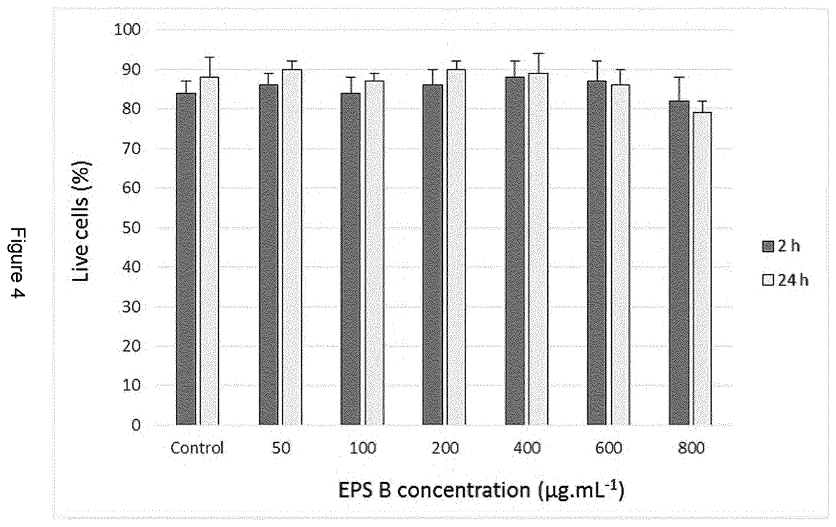

Time-dependent effects of increasing concentrations of EPS on HNEpC viability after 2 and 24 h are illustrated in FIG. 4. HNEpC cells, chosen as model because of their susceptibility, were incubated with increasing dosage of EPS for 2 hours and 24 hours, consecutively. As shown on FIG. 4, the cell survival values were identical to the control, the results indicate that the presence of EPS did not induce cytotoxicity, regardless of its concentration and its incubation duration.

### Example 8: Ex Vivo EPS Assay on HNEpC Cells

The EPS used in the example herein was obtained by fermentation of Bacillus licheniformis LP-T14 (deposit number NCIMB 43557) as described in Example 1.

The efficacy of EPS compositions to remove of two bacterial strains, i.e. Staphylococcus aureus and Pseudomonas aeruginosa, from a monolayer of human nasal epithelial cells was then assessed.

Human Nasal Epithelial Cells (HNEpC; PromoCell, Cat. Num. C-12620) were grown until 80% confluence in BEBM complete medium, as recommended by the manufacturer. HNEpC cells were washed twice with PBS and cultured with pathogenic bacteria (P. Aeruginosa or S. Aureus) at 37° C., 5% CO₂ in a medium without antibiotics for 2h. P. aeruginosa was grown in Luria Bertani broth whereas S. aureus in Tryptone soy broth. Upon overnight culture in broth, bacteria were diluted in PBS at optical density (OD600) of 0.1 and used for the experiments. Contaminated HNEpC cells were washed twice with PBS and EPS was added at 0 (Control), 50, 100, 200 and 400 μg·mL⁻¹ for 2h in BEBM complete medium. After 2 washings with PBS, a final washing has been performed with distilled H₂O for 10 minutes to kill and detach HNEpC cells and collect all residual bacteria. Supernatants (containing dead cells and bacteria) were then collected and seeded, after appropriate dilutions, on AGAR medium (cetrimide agar for P. aeruginosa and mannitol salt agar for S. aureus). Bacterial growth was analyzed upon 24 h by counting bacterial colonies (CFU) in the plates.

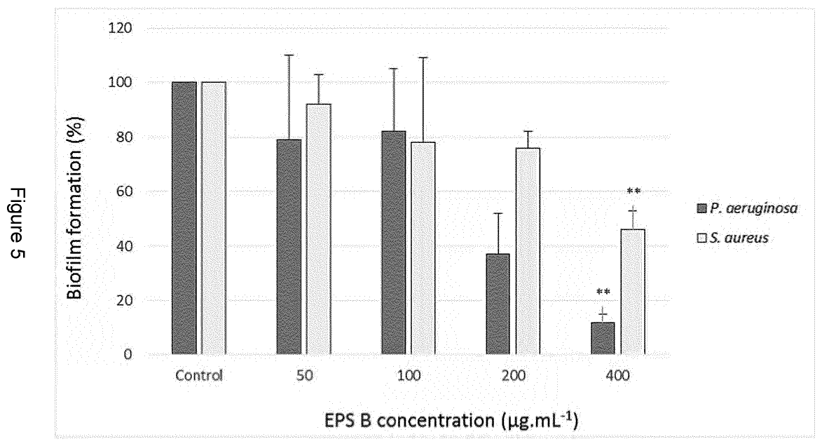

The efficacy of the EPS on the removal of P. aeruginosa and S. aureus from monolayers of human nasal epithelial cells (HNEpC) in vitro are represented in FIG. 5. These experiments were performed in triplicates (n=3) between 2-4 independent occasions (N =2-4). Statistical significance was determined by Kruskal Wallis One-Way ANOVA on ranks. A p value <0.05 was considered as statically significant. Post hoc analysis was performed using Dunn's test.

By measuring the number of pathogenic bacteria that were attached to nasal cells prior to osmotic shock (washing with deionized water), these experiments indirectly measure the effect of EPS on the detachment of pathogenic bacteria from their support, i.e., epithelial nasal cells (HNEpC). Hence, these results showed that the EPS had the capacity to limit the consequences of bacterial biofilm installation, whatever its origin, on biological surfaces. Indeed, in addition to the prophylactic effect demonstrated in Example 5 in which the formation of biofilm was impacted by the presence of EPS, it was demonstrated herein that EPS washing can have a therapeutic activity due to its pathogenic bacterial cells removal effect (FIG. 5).

### Example 9: EPS Emulsifying/Surfactant Properties

The EPS used in the example herein was obtained by fermentation of Bacillus licheniformis LP-T14 (deposit number NCIMB 43557) as described in Example 1.

The emulsifying/surfactant activity of the EPS, via a method described by Song et al. (Preparation of Calcipotriol Emulsion using bacterial Exopolysaccharides as Emulsifier for percoutaneous treatment of Psoriasis Vulgaris. Int J Mol Sci. 2019, 20;21(1) 2019) was determined. Briefly, freeze-dried EPS was resuspended in PBS to reach several concentrations ranging from 5 to 800 μg·mL⁻¹. A volume of 2.0 mL of each EPS solution was added within a glass tube with 2.0 mL vegetable oil. The heterogeneous resultant solution was then mixed for 2 min at 3000 rpm using a vortex mixer for emulsification. “Vegetable oil+PBS” and “vegetable oil+Saponin (1%)” were used as negative and positive control, respectively. The samples were placed at room temperature for 24 h without agitation prior to emulsion index evaluation (E24). E24 is represented by the height of the emulsified layer, divided by the total liquid column height, multiplied by 100.

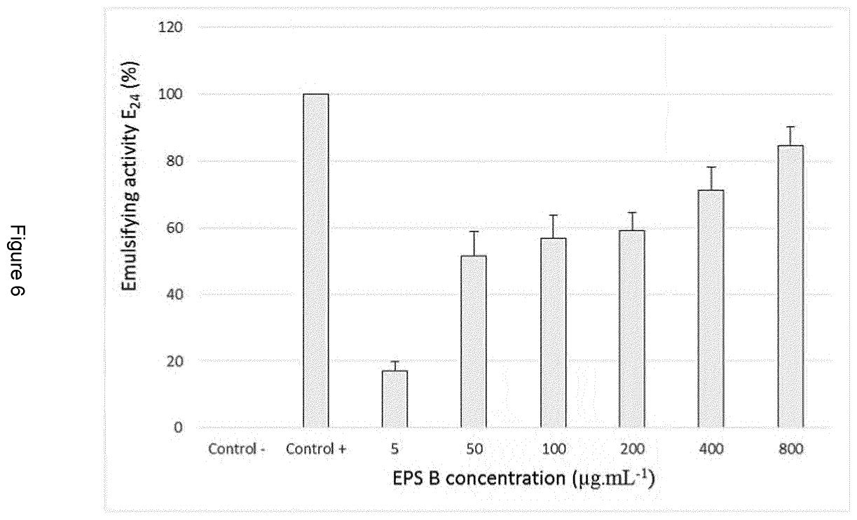

The results of three independent experiments (N=3), depicted in FIG. 6, demonstrated that EPS display a strong emulsifying activity even at low concentration (i.,e., an E24 of 50% was reached using 50 μg·mL⁻¹ only, corresponding to 0,005% EPS). Moreover, it was clearly demonstrated that higher amounts of EPS in the environment, the greater the emulsifying effect.

### Example 10: Ex Vivo Prophylactic Antiviral Activity of EPS on HNEpC Inoculated with high Dosage of Adenovirus or Rhinovirus

The EPS used in the example herein was obtained by fermentation of Bacillus licheniformis LP-T14 (deposit number NCIMB 43557) as described in Example 1.

The ability of EPS to prevent viral infection of living human nasal epithelial cells inoculated with Human Adenovirus 2 (ATCC VR-846) and Human RhinoVirus 16 (ATCC VR-283) was assessed. Briefly,

Human Nasal Epithelial Cells (HNEpC) were grown until 80% confluence in BEBM complete medium, as recommended by the manufacturer. Cells were treated with EPS at 400 μg·mL⁻¹ and incubated for 2 h at 37° C., 5% CO₂, washed with PBS and then inoculated and incubated respecting same conditions with a high Rhinovirus or Adenovirus load (1 TCID50, representing the viral dosage inducing a cytopathic effect in 50% of inoculated cells after 48 h). Alternatively, living HNEpC were infected with the same inoculum for 2 h at 37° C., 5% CO₂, washed and then treated with EPS at 400 μg·mL−1 for additional 2 h at 37° C., 5% CO₂. Untreated living cells (BEBM complete medium only) was used as positive control, whereas virus inoculated living cells without EPS treatment were used as negative control. After washing with PBS, pre-treated cells (i.e., cells treated with EPS composition prior to viral inoculation), post-treated cells (i.e., cells treated with EPS composition after viral inoculation) and controls were cultured in BEBM medium for 48 h, at 37° C., 5% CO₂. After 48 h culture, cells were collected, and viability was assessed by staining with viability dye TO-PRO-3. Percentage of TO- PRO-3 negative cells, representing viable cells in the culture, were evaluated by Symphony BD Sciences flow cytometer.

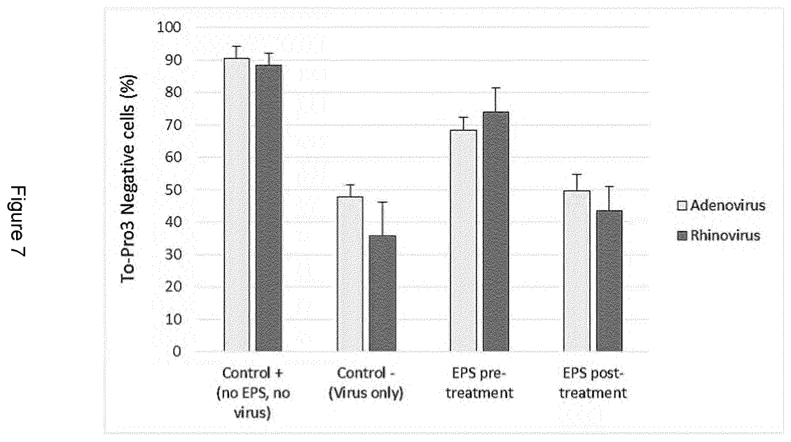

Antiviral activity of EPS (400 μg·mL⁻¹) on HNEpC infected by high dosage of adenovirus or rhinovirus is reported in FIG. 7. The negative control (viral inoculation only) shows that half of the HNEpC were affected by the viral inoculation after 48h. However, EPS pre-treatment of the cells prior to virus inoculation reduced the observed cell mortality by half vs. negative control. Such pre-treatment has thus increased cell survival after viral infection and highlights the prophylactic character of EPS against viral infections, independently from the virus considered. EPS treatment of nasal epithelial cells that have been previously inoculated with a viral load is less pronounced than for pre-treatment (FIG. 7). Without wishing to be bound to theory, such observation can be explained by the high viral load inoculation, where a large amount of viruses has been internalized within HNEpC in 2 hours, allowing rapid contamination of a large part of the cells by virions. These results indirectly demonstrated that EPS did not promote any intracellular effect (i.e., immune response triggering), but had rather an extracellular “barrier” effect that would sterically hinder the internalization of viruses. Nevertheless, it is worth mentioning that the inoculation of such a high viral load (i.e., 1 TCID50) does not reflect the reality of a viral infection, where lower virus load inoculation is involved (i.e, 0.001 TCID50).

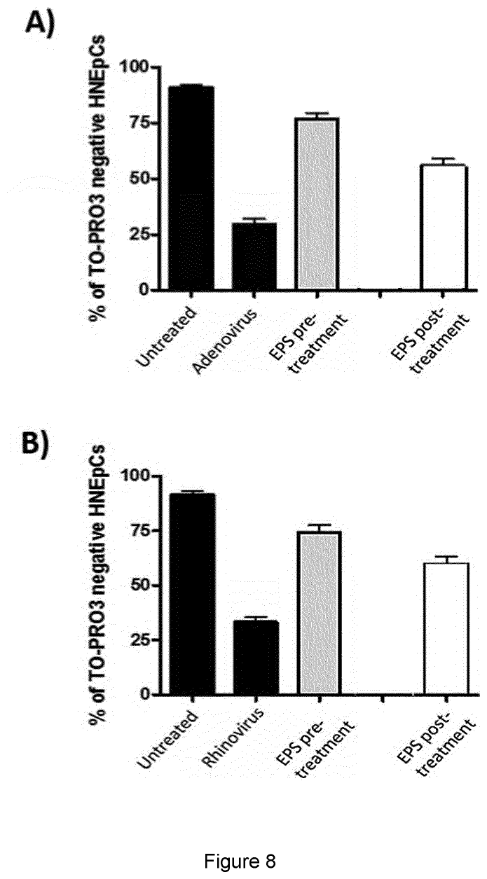

Further antiviral experiments were performed under the same conditions except that EPS concentration was lowered to 200 μg·mL⁻¹. The antiviral activity of EPS (200 μg·mL⁻¹) on HNEpC inoculated by high dosage of adenovirus or rhinovirus is reported in FIG. 8A and FIG. 8B, respectively. These results demonstrated a prophylactic antiviral activity equivalent to what has been observed for higher doses of EPS.

### Example 11: Ex Vivo Therapeutic Antiviral Activity of EPS on HNEpC Inoculated with low dosage of Adenovirus or Rhinovirus

The EPS used in the example herein was obtained by fermentation of Bacillus licheniformis LP-T14 (deposit number NCIMB 43557) as described in Example 1.

The ability of the EPS to prevent spreading of cell infection subsequent to the release of virions from cells inoculated by viral inoculum was determined. In order to be more consistent with the reality of a viral infection, these experiments were performed using lower viral load (i.e., 0.001 TCID50) of both Human Adenovirus 2 (ATCC VR-846) and Human RhinoVirus 16 (ATCC VR-283) than presented in Example 10 while extending cell culture duration up to 5 days to allow viral spreading following inoculum. It was also sought to understand whether a consistent presence of EPS is required or if an initial contact with HNEpC is sufficient enough to exert a protective effect. Hence, different cell culture conditions were performed, in which EPS were added only as pre-treatment (as within the Example 10) or further added following viral inoculum (either added once at the beginning of the 5 days culture or adding every day for 5 days). Briefly, living Human Nasal Epithelial Cells (HNEpC) were grown until 80% confluence in BEBM complete medium, as recommended by the manufacturer. i) As positive control, living HNEpC were cultured in BEBM medium, whereas the negative control involved HNEpC that were inoculated by viral inoculum (low dose, 0.001 TCID50) for 2 h, washed with PBS to get rid of viruses that were not internalized and cultured for 5 days. ii) Living HNEpC were treated with EPS at 400 μg·mL-1 for 2 h, washed with PBS and inoculated by viral inoculum (low dose, 0.001 TCID50) for 2h, washed with PBS and cultured for 5 days. iii) Living HNEpC were inoculated by viral inoculum (low dose, 0.001 TCID50) for 2h, washed with PBS and cultured for 5 days in the presence of EPS (400 μg·mL⁻¹ ), adding EPS solution in one shot at day 1. iv) Living HNEpC were inoculated by viral inoculum (low dose, 0.001 TCID50) for 2 h, washed with PBS and cultured for 5 days in the presence EPS (400 μg·mL⁻¹ ), adding EPS (400 μg·mL⁻¹) to the culture every day. After 5 days of culture, cells were collected, and their viability was assessed by TO-PRO3. Percentages of TO-PRO-3 negative cells (representing viable cells) were evaluated for each condition by Symphony BD Sciences flow cytometer.

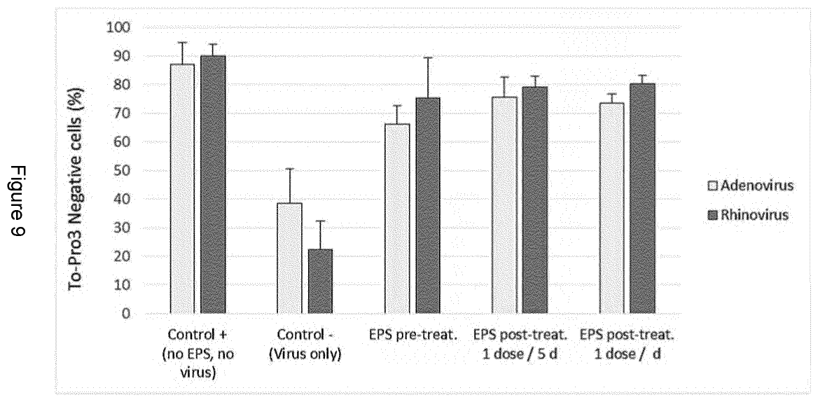

EPS antiviral activity on HNEpC inoculated by low dosage of Adenovirus or Rhinovirus is reported FIG. 9: The negative control (low dosage of viral inoculation only) showed that more than the half part of HNEpC were affected by the viral inoculation after 5 days. The prophylactic character of EPS pre-treatment (i.e., cells treated with EPS composition prior to be inoculated by a virus) against viral infection observed in Example 10 was confirmed after 5 days of culture by cell survival rates close to those of positive control. Moreover, when EPS composition was applied post-virus inoculation (i.e., post-treated cells), it is shown that viruses were not able to induce same cells mortality as for the negative control (i.e., virus without EPS treatment).

The 2 post-treatment experiments (1 EPS application in 5 days and 1 EPS application per day for 5 days) gave similar results (FIG. 9), it was demonstrated that EPS also prevents the release of virions as well as their spreading into adjacent cells. Thus, EPS provides a therapeutic effect that reduces by 50% the virulence of the viral inoculation after 5 days of incubation (FIG. 9). These results reinforce the observations of Example 10, in which an extracellular “barrier” effect that sterically hinder the internalization of viruses would be at stake here.

These two combined effects, namely prophylactic (observed with EPS pre-treatment of cells prior to viral inoculation) and therapeutic effects (observed with EPS post-treatment of cells after viral inoculation), therefore promote the extinction of the viral infection. Such

EPS administration could thus act as a support for the cellular machinery to defend itself effectively against any type of viral infection, as no specificity for the targeted viral strain has been demonstrated.

Further antiviral experiments were performed under the same except that EPS concentration was lowered to 200 μg·mL⁻¹. The antiviral activity of EPS (200 ug.mL -1) on

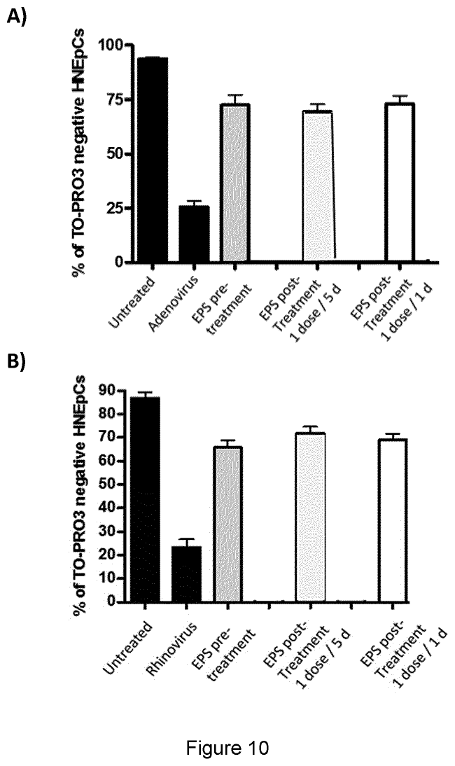

HNEpC inoculated by low dosage of adenovirus or rhinovirus is reported in FIG. 10A and FIG. 10B, respectively. These results demonstrated both prophylactic and therapeutic antiviral activity equivalent to what has been observed for higher doses of EPS.

### Example 12: Ex Vivo Therapeutic Antiviral Activity of EPS on HNEpC Infected with Low and High Dosage of Coronavirus

The EPS used in the example herein was obtained by fermentation of Bacillus licheniformis LP-T14 (deposit number NCIMB 43557) as described in Example 1.

The ability of EPS to prevent viral infection of living human nasal epithelial cells inoculated with Human Coronavirus OC43 (HCoV-OC43) at high (1 TCID50) and low (0.001 TCID50) viral loads was determined. The Human Coronavirus OC43 (HCoV-OC43) used in the study herein is the most commonly associated with human infections accounting for 5 to 30% of human respiratory tract infections. (Lau SKP et. al., 2011, Journal of Virology). Briefly, human Nasal Epithelial Cells (HNEpC) were grown until 80% confluence in BEBM complete medium, as recommended by the manufacturer. Cells were treated with EPS at 200-400 μg·mL⁻¹ and incubated for 2 h at 37° C., 5% CO₂, washed with PBS and then inoculated and incubated respecting same conditions with coronavirus dosage (either 1 TCID50, or 0.001 TCID50). Alternatively, living HNEpC were inoculated with the same inoculum for 2 h at 37° C., 5% CO₂. washed and then treated with EPS at 200-400 μg·mL⁻¹ for additional 2 h at 37° C., 5% CO₂. Untreated living cells (BEBM complete medium only) were used as positive control, whereas virus inoculated living cells without EPS treatment were used as negative control. After washing with PBS, pre-treated cells (i.e., cells treated with EPS composition prior to viral infection), post-treated cells (i.e., cells treated with EPS composition after viral infection) and controls were cultured in BEBM medium for 48 h, at 37° C., 5% CO₂. After 48 h culture, cells were collected, and viability was assessed by staining with viability dye TO-PRO-3. Percentage of TO-PRO-3 negative cells, representing viable cells in the culture, were evaluated by Symphony BD Sciences flow cytometer.

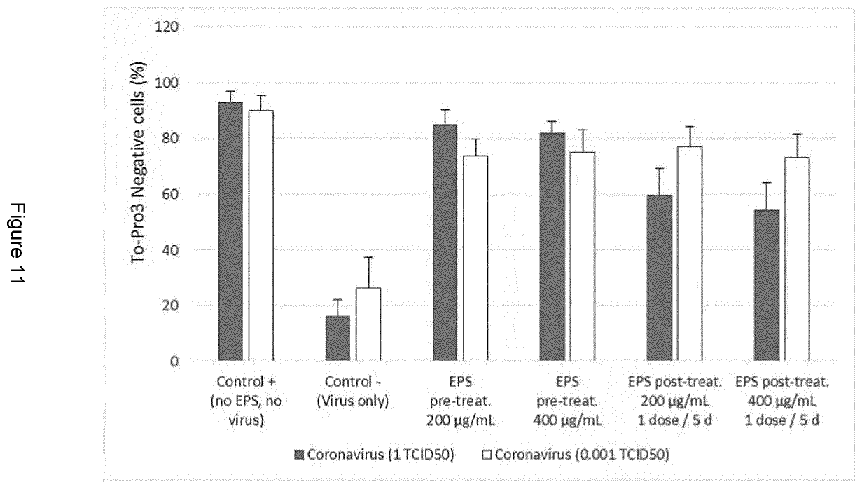

Antiviral activity of EPS (200-400 μg·mL⁻¹ ) on HNEpC inoculated with either high or low dosage of coronaviruses is reported in FIG. 11. These results demonstrated that the EPS provided: i) a prophylactic antiviral activity as described in the Example 10 herein and ii) a therapeutic antiviral activity as described in the Example 11 herein, independently from the virus considered.

### Example 13: EPS Formulation—Nasal Formulations

The EPS used in the example herein was obtained by fermentation of Bacillus licheniformis LP-T14 (deposit number NCIMB 43557) as described in Example 1.

Nasal formulation 1: Seawater and desalinated Seawater were mixed in a cleaned bulk tank at room temperature until reaching the desired salinity (0.9% or 2.2% or 2.7% according to formula). A volume of this bulk solution (10 L) is collected to solubilize the freeze-dried EPS of the present disclosure, reaching a desired EPS concentration of 0.02% or 0.04% corresponding to 200 μg/mL or 400 μg/mL (depending on the desired formula). Both EPS and bulk solution were homogenized for at least 30 min and then filtered on 0.22 μm. This operation was repeated once, prior to filling the nasal spray can.

Nasal formulation 2: Sodium chloride was dissolved in water at room temperature and in a cleaned bulk tank to reach the desired salt concentration of 0.9%, 2.2% or 2.7% (depending on the desired formula). EPS of the present disclosure were then dissolved within the bulk solution above to reach the desired EPS concentration of 0.02% or 0.04% corresponding to 200 μg/mL or 400 μg/mL (depending on the desired formula). Both EPS and bulk solution were homogenized for at least 30 min and then filtered on 0.22 μm. This operation was repeated once, prior to filling the nasal spray can.

### Example 14: EPS Formulation—Topic Formulation (Cream)

The EPS used in the example herein was obtained by fermentation of Bacillus licheniformis LP-T14 (deposit number NCIMB 43557) as described in Example 1.

Polyacrylate crosspolymer-6 (1.5% w/w) and water (63.0% w/w) were mixed thoroughly prior to xanthan gum (4.0 w/w) and glycerin (2.0) addition. The resulting mixture (Phase A) was warmed up to 70-75° C. under continuous mixing. A mixture of sucrose polystearate (3.0% w/w), glyceryl dibehenate (1.7% w/w), hydrogenated jojoba oil (21.0% w/w) was heated to 70-75° C. and then added to Phase A under continuous mixing, giving Phase B. Phase B was mixed for emulsification for 20 min at 70-75° C. and then, the emulsified Phase B was allowed to cool down to 30° C. EPS (0.02-0.04% w/w), Vitamin E acetate and perfume were added to the emulsified Phase B at 30° C. under constant agitation. The pH of the resulting mixture was adjusted to pH 4.5-5.0 with a citric acid solution in water (qsp.).

### Example 15: Pharmacological/Immunological Character of the EPS Disclosed Herein

The EPS used in the example herein was obtained by fermentation of Bacillus licheniformis LP-T14 (deposit number NCIMB 43557) as described in Example 1.

As it has been reported that EPS produced by marine bacteria can induce selected pattern of regulatory cytokines in leucocytes and tissue cells (Okutani K. et al. Bull Jpn Soc Sci Fish 1984;50:1035-7), the purpose of these assays was to determine to the extent to which the EPS of the present disclosure would be able to induce an immune response on the cells which the EPS would be in contact with. Thus, as two different systems are likely to be susceptible of modifications when in contact with substances derived from bacteria, i.e., epithelial cells and leukocytes of the innate immune system, the assays herein were performed on Human Nasal Epithelial Cells (HNEpC) and human monocyte-derived Dendritic Cells (DC), respectively.

More particularly, the following sets of experiments were performed to evaluate i) the production of key cytokines involved in inflammatory pathway, such as IL-6 and IL-8, following culture with EPS samples with HNEpC, and ii) the activation status of DC following EPS stimulation by assessing the expression of the most important activation/maturation markers, such as CD83, CD40 and CD25 and the production of inflammatory cytokines (IL-6 and IL-8).

### i) Effect of the EPS of the Present Disclosure on HNEpC:

Human Nasal Epithelial Cells (HNEpC) were grown until confluence in BEBM complete medium and then stimulated with an EPS sample (400 μg/mL) for 24 h. Cells left unstimulated or stimulated with IL-1β (50 ng/mL) were employed as negative and positive controls, respectively. Monensin and Brefeldin were used as GolgiStop (Protein Transport Inhibitor) and added for the last 12 h of culture. Cells were collected, fixed in 1% paraformaldehyde, permeabilized with saponin 0.1% (Sigma-Aldrich) in PBS and stained with the phycoerythrin (PE)—conjugated anti-IL-8 and anti-IL-6 antibody (BD Bioscience) and then analyzed by flow cytometry.

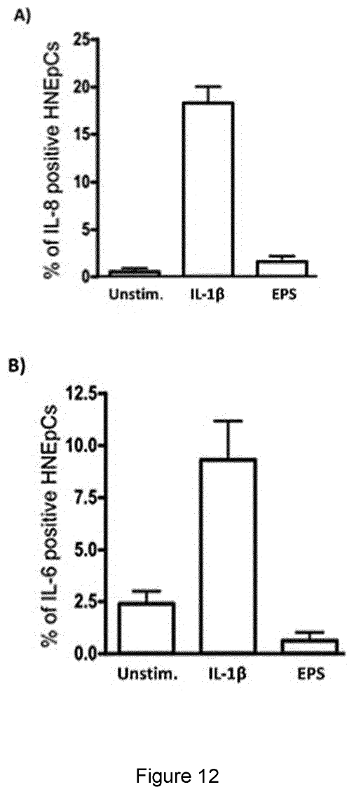

These two cytokines are the main mediators involved in the inflammatory pathway and in the recruitment of immune cells able to start a phlogistic event. As depicted in FIG. 12A & FIG. 12B, there was no production of IL-6 and IL-8 by HNEpC following 24 h stimulation with the EPS of the present disclosure, when compared to unstimulated HNEpC. Remarkably, despite HNEpC were competent to produce these cytokines when appropriately stimulated by a prototypical inflammatory molecule (i.e., IL-1β), no cytokine production was observed upon EPS exposure. Therefore, these results indicated that the EPS of the present disclosure is not able to induce an inflammatory response.

### ii) Effect of the EPS of the Present Disclosure on DC:

Peripheral blood mononuclear cells (PBMCs) were isolated from two healthy donors by Ficoll Hypaque density gradient centrifugation. Monocytes were isolated by adherence to plastic surfaces for 45 min at 37° C., 5% CO₂. Subsequently, non-adherent cells were removed by washing with phosphate-buffered saline (PBS). Cells obtained were cultured in complete RPMI medium supplemented in the presence of IL-4 (20 ng/mL, Miltenyi Biotec, Germany) and GM-CSF (25 ng/mL, Sargramostim). Following a 6 day of culture, non-adherent cells displayed the CD⁻/CD11c⁺/CD83⁻ phenotype, which is characteristic of immature dendritic cells (DC).

DC were stimulated with the EPS disclosed herein (400 μg/mL) for 24 h. Cells left unstimulated or stimulated with lipopolysaccharide (LPS, 1 μg/mL) were employed as negative and positive controls, respectively. For cytokine production analysis, Monensin and Brefeldin were used as GolgiStop and added to DC culture for the last 12 h. A) DC activation: After 24 h, DC were collected and stained with anti-CD83 (Anti-CD83-PE), anti-CD40 (Anti-CD40-AlexaFluor 647) and anti-CD25 (Anti-CD25-PacificBlue) monoclonal antibodies at 4° C. for 20 minutes. Cells were washed with PBS and then analyzed for the level of surface expression of the indicated activation molecules by flow cytometry. B) Cytokine production: DC were collected, fixed in 1% paraformaldehyde, permeabilized with saponin 0.1% (sigma-Aldrich) in PBS, stained with the phycoerythrin (PE)-conjugated anti-IL-8 and anti-IL-6 antibody (BD Bioscience) and analyzed by flow cytometry.

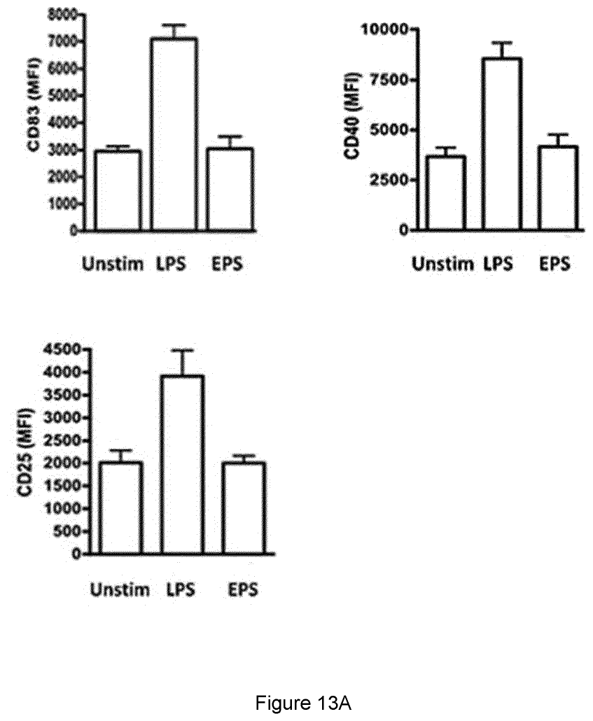

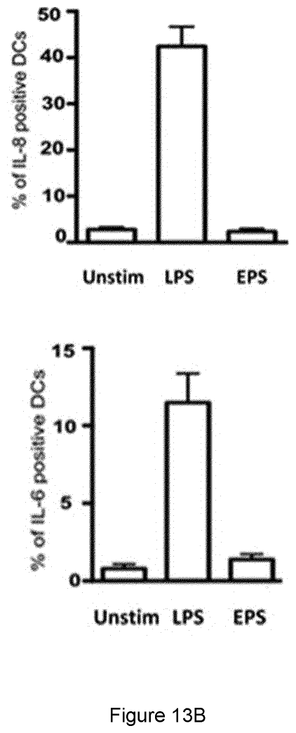

The effects of the EPS of the present disclosure on DC were assessed evaluating both DC activation by analyzing the expression level of CD83, CD40 and CD25 as Mean of Fluorescence Intensity (MFI), as represented in FIG. 13A. Analysis of DC, following EPS stimulation, revealed that the EPS of the present disclosure had no effect on DC maturation, as demonstrated by the very low expression level of CD83, CD40 and CD25, which remained comparable to unstimulated DC.

The effects of the EPS of the present disclosure on DC and their cytokine production, i.e., IL-8 and IL-6, following 24h of stimulation was evaluated as percentage of cytokine-producing cells, as depicted in FIG. 13B. There was no production of IL-6 and IL-β by DC following 24 h stimulation with the ESP, when compared to unstimulated DC.

It is noteworthy that both human cell types used in these experimental systems (HNEpC and DC) are well equipped with Pattern Recognition Receptors (PRR) able to sense the presence of Pathogen-Associated-Molecular Patterns (PAMP), including bacteria. DC are extremely competent in sensing potential PAMP in order to start an inflammatory process. In addition, it should be noted that LPS derived from gram-negative bacteria and used as positive control in the DC experiments, was effective at 1 μg/ml, i.e., at a concentration 400 folds lower than the tested EPS concentration (FIG. 13). Therefore, it has been demonstrated that the EPS of the present disclosure lacks the ability to induce an immune (cellular and/or humoral) response.

While the invention has been described in connection with specific embodiments thereof, it will be understood that the scope of the claims should not be limited by the preferred embodiments set forth in the examples, but should be given the broadest interpretation consistent with the description as a whole.

Further aspects and embodiments of the present invention are set out in the following numbered paragraphs:
1. A composition comprising at least one isolated exopolysaccharide (EPS) originating from a marine bacteria and a suitable carrier, wherein the at least one isolated EPS having an average molecular weight ranging from 40 to 4000 kDa, and wherein the at least one isolated EPS is obtainable by fermentation of the marine bacteria.
2. The composition of paragraph 1, wherein the composition comprises at least two exopolysaccharides each independently having an average molecular weight ranging from 40 to 4000 kDa.
3. The composition of paragraph 1 or 2, wherein the average molecular weight of the EPS is from 40 to 2000 kDa, optionally from 100 to 1400 kDa.
4. The composition of any one of paragraphs paragraph 1 to 3, wherein the marine bacteria is a gram-positive thermophilic bacteria, optionally wherein the gram-positivethermophilic bacteria is Bacillus licheniformis, preferably wherein said gram-positive thermophilic bacteria is Bacillus licheniformis LP-T14 which was deposited with The National Collection of Industrial, Food and Marine Bacteria (NCIMB) on 27 Jan. 2020 under deposit number NCIMB 43557.
5. The composition of any one of paragraphs 1 to 4, wherein:
   said composition is an oral composition, a nasal composition, a topical composition, a transdermal composition, an ophthalmic composition or a composition formulated for treating a medical device, preferably wherein said composition is a nasal composition; and/or
   (ii) said carrier is saline solution.
6. A nasal delivery system comprising at least one isolated exopolysaccharide (EPS) originating from a marine bacteria, wherein the at least one isolated EPS has an average molecular weight ranging from 40 to 4000 kDa, and wherein the at least one isolated EPS is obtainable by fermentation of the marine bacteria.
7. The nasal delivery system of paragraph 6, wherein:
   (i) the average molecular weight of the at least one isolated EPS is from 40 to 2000 kDa, optionally from 100 to 1400 kDa; and/or
   (ii) the marine bacteria is a gram-positive thermophilic bacteria, optionally wherein the gram-positive thermophilic bacteria is Bacillus licheniformis, preferably wherein said gram-positive thermophilic bacteria is Bacillus licheniformis LP-T14 which is deposited with the NCIMB under deposit number NCIMB 43557;
   (iii) the nasal delivery system delivers the at least one isolated EPS as nose drops, a liquid spray, a dried spray, a gel or an ointment;
   (iv) the at least one isolated EPS is formulated in a composition that further comprises a saline solution;
   (v) the nasal delivery system is for treating and/or preventing a microbial pathogen infection in a patient in need thereof; and/or
   (vi) the nasal delivery system is for attenuating the virulence of a microbial pathogen by inhibiting or reducing colonization by the microbial pathogen of the nasal cavity of a patient in need thereof.
8. A non-therapeutic use of a composition as defined in any one of paragraphs 1 to 5 for treating a non-biological surface to prevent or reduce the colonization of the surface by a microbial pathogen, optionally wherein the surface is a surface of a medical device.
9. A composition as defined in any one of paragraphs 1 to 5 or a nasal delivery system as defined in paragraph 6 or 7 for use in a method for treating and/or preventing a microbial pathogen infection in a patient in need thereof.
10. A composition as defined in any one of paragraphs 1 to 5 or a nasal delivery system as defined in paragraph 6 or 7 for use in a method for attenuating the virulence of a microbial pathogen.
11. A method of treating and/or preventing a microbial pathogen infection in a patient in need thereof, the method comprising administering to the patient an effective amount of a composition as defined in any one of paragraphs 1 to 5.
12. A method for attenuating the virulence of a microbial pathogen infection in a patient in need thereof, the method comprising the administration of an effective amount of a composition as defined in any one of paragraphs 1 to 5 to the patient.
13. The composition for use according to paragraph 9 or 10 or the method of paragraph 11 or 12, wherein:
   (i) the composition is administered to the patient prior, during, and/or after the microbial pathogen infection, thereby preventing, inhibiting, or reducing colonization of the microbial pathogen at least one biological tissue of the patient and/or the internalization of the microbial pathogen, optionally wherein the at least one biological tissue is selected from the oral cavity, the nasal cavity, the respiratory tract, the throat, the ears, the ophthalmic region, the urogenital tract, the skin, the scalp, the hairs, the nails, and combinations thereof, preferably wherein the at least one biological tissue is the nasal cavity; and/or
   (ii) the microbial pathogen is a bacterial pathogen and administration of the composition to the patient attenuates the virulence of the bacterial pathogen infection by reducing or inhibiting early bacterial biofilm formation and/or disrupting early bacterial biofilm; or
   (iii) the microbial pathogen is a viral pathogen and administration of the composition to the patient attenuates the virulence of a viral pathogen infection by reducing the mortality of patient cells infected with the viral pathogen as compared to equivalent untreated cells, by preventing or reducing the release of virions from infected patient cells and/or by preventing or reducing the infection of uninfected adjacent patient cells.
14. The nasal delivery system of paragraph 6 or 7, the use of paragraph 8, the composition for use according to paragraph 9, 10 or 13, or the method of any one of paragraphs 11 to 13, wherein the microbial pathogen is:
   (i) a bacterial pathogen optionally selected from the Cutibacterium, Haemophilus, Klebsiella, Moraxella, Pseudomonas, Staphylococcus and/or Streptococcus genera, preferably wherein the bacterial pathogen is Cutibacterium acnes, Haemophilus influenzae, Klebsiella pneumoniae, Moraxella catharalis, Pseudomonas aeruginosa, Staphylococcus aureus, Streptococcus pneumoniae or Streptococcus mutans species, or a combination thereof;
   (ii) a viral pathogen optionally selected from Influenza A, Influenza A subtype H1N1/2009/pdm09, Influenza A subtype H1, Influenza A subtype H3, Influenza B (Orthomyxovirus), Coronavirus 229E, Coronavirus HKU1, Coronavirus NL63, Coronavirus OC43, SARS-CoV-2 (Coronavirus), Parainfluenza Virus 1, Parainfluenza Virus 2, Parainfluenza Virus 3, Parainfluenza Virus 4, Respiratory Syncytial Virus NB, Human Metapneumovirus NB (Paramyxovirus), Adenovirus or Rhinovirus/Enterovirus (Picornavirus), or a combination thereof;
   (iii) a fungal pathogen; or
   (iv) a mixture of one or more of (i), (ii) and (iii).
15. The composition of any one of paragraphs 1 to 5, the use of paragraph 8, the composition for use according to any one of paragraphs 9, 10, 13 and 14, or the method of any one of paragraphs 11 to 14, wherein the amount of the at least one EPS in the composition based on the total weight of the composition is in the range of 0.00005-0.5% w/w, preferably 0.0001-0.1% w/w, more preferably 0.0005-0.05% w/w.
16. A method of formulating a nasal composition for attenuating the virulence of a microbial pathogen infection by inhibiting or reducing colonization by a microbial pathogen within the nasal cavity, wherein the method comprises the step of mixing at least one isolated exopolysaccharide (EPS) originating from a marine bacteria, with a saline solution and optionally a suitable carrier to obtain a nasal composition comprising salt at a concentration of 0.1% to 10% w/w, preferably 0.5% to 5% w/w, more preferably 0.7% to 3% w/w.

1. An isolated exopolysaccharide (EPS) having a weight average molecular weight (Mw) ranging from 40 to 4000 kDa, wherein the EPS is obtained or obtainable by fermentation of a marine bacteria.

2. The isolated EPS of claim 1 having a weight average molecular weight (Mw) ranging from 40 to 2000 kDa, 40 to 1400 kDa, 40 to 1000 kDa, 40 to 500 kDa, 40 to 400 kDa, 40 to 300 kDa, 40 to 200 kDa, 40 to 150 kDa, or 40 to 100 kDa, preferably 40 to 150 kDa.

3. The isolated EPS of claim 1 or 2 having an Mw ranging from 40 to 150 kDa and optionally a number average molecular weight (Mn) ranging from 26 to 100 kDa and/or a polydispersity index (Mw/Mn) ranging from 1.2 to 1.8.

4. The isolated EPS of any one of claims 1 to 3 comprising 30-90% of neutral glycosyl units, 10-70% of amino glycosyl units, and 0-15% of acidic glycosyl units with respect to the total number of glycosyl units of said EPS.

5. The isolated EPS of any one of claims 1 to 4 comprising mannose, galactose, glucose, N-acetyl galactosamine and N-acetyl glucosamine.

6. The isolated EPS of any one of claims 1 to 5 being substantially free of ribose, arabinose, rhamnose, fructose and/or fucose.

7. The isolated EPS of any one of claims 1 to 6, wherein the marine bacteria is a gram-positive thermophilic bacteria, preferably wherein the gram-positive thermophilic bacteria is Bacillus licheniformis, more preferably wherein said gram-positive thermophilic bacteria is Bacillus licheniformis LP-T14 which was deposited with The National Collection of Industrial, Food and Marine Bacteria (NCIMB) on 27 Jan. 2020 under deposit number NCIMB 43557.

8. The isolated EPS of any one of claims 1 to 7 substantially lacking the ability to induce an immune response.

9. A process for obtaining an isolated exopolysaccharide (EPS) having a weight average molecular weight (Mw) ranging from 40 to 4000 kDa comprising the steps of:
culturing a marine bacteria in a first culture medium to obtain a cultured marine bacteria;
fermenting the cultured marine bacteria in a fermentation medium;
heat treating the fermentation medium;
centrifuging the fermentation medium to obtain a supernatant; and
filtering the supernatant to obtain the isolated EPS.

10. The process of claim 9 wherein:
the isolated EPS is as defined in any one of claims 2 to 6;
the marine bacteria is a gram-positive thermophilic bacteria, preferably wherein the gram-positive thermophilic bacteria is Bacillus licheniformis, more preferably wherein said gram-positive thermophilic bacteria is Bacillus licheniformis LP-T14 which was deposited with The National Collection of Industrial, Food and Marine Bacteria (NCIMB) on 27 Jan. 2020 under deposit number NCIMB 43557;
the fermentation medium comprising sea salts (40 g/L), tryptone (6 g/L), yeast extract (6 g/L), antifoam (0.33 mL/L), dextrose (12 g/L) and deionized H₂ O (qsp.);
the fermentation is performed at pH 5-8 for a period of 20-40 h at 40° C., under 30-50% oxygenation;
the heat treatment of fermentation medium is performed by heating the fermentation medium for 1 h at 85° C.;
the centrifugation of the fermentation medium is performed at 14000 g using a disk stack centrifuge with a flow rate of 200-800 L/h; and/or
the filtration of supernatant comprises successive filtration using 1.60 to 0.22 μm filtration steps and/or ultrafiltration using 10 to 100 kDa cut-off filter cartridges.

11. The process of claim 9 or 10, which further comprises free-drying the isolated EPS to obtain a freeze-dried EPS, optionally wherein the free-drying is performed at −20° C. for at least 16 hours.

12. A composition comprising at least one isolated exopolysaccharide (EPS) according to any one of claims 1 to 8 or at least one isolated EPS obtained or obtainable by the process of any one of claims 9 to 11 and a suitable carrier.

13. The composition of claim 12 being an oral composition, a nasal composition, a topical composition, a transdermal composition, an ophthalmic composition or a composition formulated for applying on a medical device.

14. A nasal delivery system comprising at least one isolated exopolysaccharide (EPS) according to any one of claims 1 to 8 or at least one isolated EPS obtained or obtainable by the process of any one of claims 9 to 11.

15. The nasal delivery system of claim 14, wherein:
the nasal delivery system delivers the at least one isolated EPS as nose drops, a liquid spray, a dried spray, a gel or an ointment;
the at least one isolated EPS is formulated in a composition that further comprises a saline solution;
the nasal delivery system is for treating and/or preventing a microbial pathogen infection in a subject in need thereof; and/or
the nasal delivery system is for attenuating the virulence of a microbial pathogen by inhibiting or reducing colonization by the microbial pathogen of the nasal cavity of a subject in need thereof.

16. A non-therapeutic use of an isolated EPS as defined in any one claims 1 to 8, an isolated EPS obtained or obtainable by the process of any one of claims 9 to 11, or a composition as defined in claim 12 or 13 for applying on a non-biological surface to prevent or reduce the colonization of the surface by a microbial pathogen, optionally wherein the surface is a surface of a medical device.

17. An isolated exopolysaccharide (EPS) as defined in any one of claims 1 to 8, an isolated EPS obtained or obtainable by the process of any one of claims 9 to 11, a composition as defined in claim 12 or 13, or a nasal delivery system as defined in claim 14 or 15 for use in a method for treating and/or preventing a microbial pathogen infection in a subject in need thereof.

18. An isolated exopolysaccharide (EPS) as defined in any one of claims 1 to 8, an isolated EPS obtained or obtainable by the process of any one of claims 9 to 11, a composition as defined in claim 12 or 13, or a nasal delivery system as defined in claim 14 or 15 for use in a method for attenuating the virulence of a microbial pathogen.

19. A method of treating and/or preventing a microbial pathogen infection in a subject in need thereof, the method comprising administering to the patient an effective amount of an isolated exopolysaccharide (EPS) as defined in any one of claims 1 to 8, an isolated EPS obtained or obtainable by the process of any one of claims 9 to 11, or a composition as defined in claim 12 or 13.

20. A method for attenuating the virulence of a microbial pathogen infection in a subject in need thereof, the method comprising the administration of an effective amount of an isolated exopolysaccharide (EPS) as defined in any one of claims 1 to 8, an isolated EPS obtained or obtainable by the process of any one of claim 9 to 11, or a composition as defined in claim 12 or 13 to the subject.

21. The isolated EPS or the composition for use according to claim 17 or 18, or the method of claim 19 or 20, wherein:
the isolated EPS or the composition is administered to the subject prior, during, and/or after the microbial pathogen infection, thereby preventing, inhibiting, or reducing colonization of the microbial pathogen on at least one biological tissue of the subject and/or the internalization of the microbial pathogen; wherein the at least one biological tissue comprises an epithelium such as an oral cavity, a nasal cavity, a respiratory tract, a throat, an ear, an ophthalmic region, a urogenital tract, a skin, a scalp, a hair, a nail, and combinations thereof;
the microbial pathogen is a bacterial pathogen and administration of the isolated EPS or the composition to the subject attenuates the virulence of the bacterial pathogen infection by reducing or inhibiting early bacterial biofilm formation and/or disrupting early bacterial biofilm; and/or
the microbial pathogen is a viral pathogen and administration of the isolated EPS or the composition to the subject attenuates the virulence of a viral pathogen infection by reducing the mortality of subject cells inoculated with the viral pathogen as compared to equivalent untreated cells, by preventing or reducing the release of virions from inoculated subject cells and/or by preventing or reducing the infection of uninoculated adjacent subject cells.

22. The nasal delivery system of claim 15, the use of claim 16, the isolated EPS or composition for use according to any one of claim 17, 18 or 21, or the method of any one of claims 19 to 21, wherein the microbial pathogen is at least one of:
a bacterial pathogen optionally selected from the Cutibacterium, Haemophilus, Klebsiella, Moraxella, Pseudomonas, Staphylococcus and/or Streptococcus genera; preferably the bacterial pathogen is Cutibacterium acnes, Haemophilus influenzae, Klebsiella pneumoniae, Moraxella catharalis, Pseudomonas aeruginosa, Staphylococcus aureus, Streptococcus pneumoniae and/or Streptococcus mutans species;
a viral pathogen optionally selected from Influenza A, Influenza A subtype H1N1/2009/pdm09, Influenza A subtype H1, Influenza A subtype H3, Influenza B (Orthomyxovirus), Coronavirus 229E, Coronavirus HKU1, Coronavirus NL63, Coronavirus OC43, SARS-CoV-2 (Coronavirus), Parainfluenza Virus 1, Parainfluenza Virus 2, Parainfluenza Virus 3, Parainfluenza Virus 4, Respiratory Syncytial Virus A/B, Human Metapneumovirus NB (Paramyxovirus), Adenovirus and/or Rhinovirus/Enterovirus (Picornavirus); or
a fungal pathogen.

23. The nasal delivery system, use, isolated EPS for use, composition for use, or method according to claim 22, wherein the microbial pathogen is a bacterial pathogen selected from Haemophilus influenzae, Klebsiella pneumoniae, Pseudomonas aeruginosa, Staphylococcus aureus, Streptococcus pneumoniae, and/or Streptococcus mutans species.

24. The nasal delivery system, use, isolated EPS for use, composition for use, or method according to claim 22, wherein the microbial pathogen is a viral pathogen selected from Coronavirus OC43, adenovirus, and/or Rhinovirus/Enterovirus (Picornavirus).

25. The composition of claim 12 or 13, the use of claim 16, the composition for use according to any one of claims 17, 18 and 21 to 24, or the method of any one of claims 19 to 24, wherein the amount of the at least one isolated EPS in the composition based on the total weight of the composition is in the range of 0.00005-0.5% w/w, preferably 0.0001-0.1% w/w, or more preferably 0.0005-0.05w/w.

26. A method of formulating a nasal composition for attenuating the virulence of a microbial pathogen infection by inhibiting or reducing colonization by a microbial pathogen within the nasal cavity, wherein the method comprises the step of mixing at least one isolated exopolysaccharide (EPS) originating from a marine bacteria, with a saline solution and optionally a suitable carrier to obtain a nasal composition comprising salt at a concentration of 0.1% to 10% w/w, preferably 0.5% to 5% w/w, more preferably 0.7% to 3% w/w.

27. The method of claim 26, wherein the at least one isolated EPS is as defined in any one of claims 1 to 8.

28. The method of claim 27 or 28, wherein the salt concentration is about 0.9% w/w, about 2.2% or about 2.7% w/w.
